(19) United States
(12) Patent Application Publication
(10) Pub. No.: US 2023/0390403 A1
(43) Pub. Date: Dec. 7, 2023

(54) COMPOSITIONS AND METHODS FOR DRUG DELIVERY

(71) Applicant: North Carolina State University, Raleigh, NC (US)

(72) Inventors: Yevgeny Brudno, Raleigh, NC (US); Sharda Pandit, Raleigh, NC (US); Sandeep Palvai, Raleigh, NC (US); Tiffany Ferrell, Raleigh, NC (US); Joshua G. Pierce, Raleigh, NC (US); Nicholas Massaro, Raleigh, NC (US); Chris Moody, Raleigh, NC (US)

(21) Appl. No.: 18/032,149

(22) PCT Filed: Oct. 14, 2021
(86) PCT No.: PCT/US2021/055014

§ 371 Date: Apr. 14, 2023

Related U.S. Application Data
(60) Provisional application No. 63/091,642, filed on Oct. 14, 2020.

(51) Int. Cl. A61K 47/54; A61P 35/00

(52) U.S. Cl. CPC A61K 47/545; A61P 35/00

(57) ABSTRACT

Disclosed herein are methods for drug delivery, as well as kits for drug delivery.

### STATEMENT REGARDING FEDERALLY SPONSORED RESEARCH OR DEVELOPMENT

This invention was made with government support under grant number R21 CA246414 awarded by the National Institutes of Health. The government has certain rights in the invention.

### BACKGROUND

Local drug presentation made possible by drug-eluting depots has provided benefits in a vast array of diseases, including in cancer, microbial infection and in wound healing. Drug-eluting depots provide sustained drug release of therapeutics directly at disease sites with tunable kinetics, obviate the need for drugs to access disease sites from the circulation and eliminate the side effects associated with systemic therapy. These depot systems often take the form of injectable hydrogels and microparticles. Unfortunately, in many application, implantable or injectable drug depots struggle to access deep into tissues because they require injection of viscous solutions or result in the formation of a bulk material at target tissues. Of particular difficulty are applications that require injection into stiff, fibrous tissue or injection into organs that cannot accommodate increases pressures, such as the brain.

### SUMMARY

Disclosed are methods for delivering an active agent to a target tissue in a subject. The methods can comprise contacting the target tissue with a compound defined by Formula I

X-L¹-A Formula I

wherein X represents a tissue binding moiety; L¹ is absent, or represents a linking group; and A represents an active agent.

The tissue binding moiety comprises a functional group capable of chemically reacting with the target tissue to form a covalent bond. In certain embodiments, the tissue binding moiety can bind with the extracellular matrix in the target tissue. In some cases, the tissue binding can non-covalently associate with and bind to the target tissue. In some embodiments, the tissue binding moiety can comprise an antibody. In other embodiments, the tissue binding moiety can comprise a lipid which inserts into a cell membrane.

In some embodiments, the tissue binding moiety can comprise a functional group capable of chemically reacting with a functional group in a peptide to form a covalent bond. In certain embodiments, the tissue binding moiety comprises a functional group capable of chemically reacting with an amine group in a peptide to form a covalent bond, such as a sulfo-hydroxysuccinimidyl (sNHS) group. In certain embodiments, the tissue binding moiety comprises a functional group capable of chemically reacting with a thiol group in a peptide to form a covalent bond, such as a maleimide group. In certain embodiments, the tissue binding moiety comprises a functional group capable of chemically reacting with the extracellular matrix in the target tissue (e.g., a protein present in the extracellular matrix, such as collagen) to form a covalent bond.

In some embodiments, L¹ is absent. In other embodiments, L¹ is present. In some embodiments, L¹ represents a cleavable linker (e.g., a hydrolysable linker, an enzymatically cleavable linker, a photocleavable linker, or a click cleavable linker).

In some embodiments, the active agent can comprise a diagnostic agent. In other embodiments, the active agent can comprise a therapeutic agent. In certain embodiments, the therapeutic agent can comprise an anti-cancer drug, a drug that promotes wound healing, a drug that promotes vascularization, a drug that treats or prevents infection, a drug that prevent restenosis, a drug that reduces macular degeneration, a drug that prevents immunological rejection, a drug that prevents thrombosis, or a drug that treats inflammation.

In some embodiments, contacting the target tissue with the compound can comprise injecting or infusing a pharmaceutical composition comprising the compound into the target tissue.

### DESCRIPTION OF DRAWINGS

FIG. 1 is a schematic illustration showing ECM anchoring of therapeutic agent to form local drug depots in the target tissues. Left: injections of therapeutic molecule modified with activated NHS esters in the target tissue. Middle: formation of ECM anchored local drug depots. Right: release of active therapeutic motifs in the target tissues following cleavage of a linker.

FIG. 2 schematically illustrates the results modeling of the anchoring to tumor ECM with intratumoral fluid flow using an azide-containing agent as a model. The figure includes a schematic diagram of NHS-ester injection, aminolysis, and hydrolysis as well as COMSOL Multi-physics model parameters. The bottom left includes a 0D model estimating the change in the concentration of the injected azide-sNHS ester, hydrolyzed species, and ECM-anchored azides over time. The expected reaction kinetics is further layered on a three-dimensional (3D) space-dependent model that leads to the results in the plot on the bottom right. The plot on the bottom right shows the number of anchored azides over mm from the center of the infusion needle in the tumor

FIGS. 3A and 3B show the distribution of anchoring for a fluorescent reactive NHS ester throughout a tissue, in this case a pancreatic tumor, by evaluating the fluorescent NHS-ester and extracellular matrix co-localization within the pancreatic tumor. FIG. 3A shows the whole tumor of fluorophore-NHS-injected tumors stained for extracellular proteins with picrosirius red. FIG. 3B shows zoom-in images of the boxed area of fluorophore-NHS-injected tumors stained for extracellular proteins with picrosirius red. Pancreatic KPC 4662 tumors were injected intratumorally with AF647 NHS ester. After 24 h, tumors were excised, fixed, sectioned, and stained with picrosirius red. The scale bars for FIG. 3A and FIG. 3B are 2 mm and 100 μm, respectively.

FIG. 4 illustrates the synthesis ECM-anchoring PTX-sNHS. First, PTX was conjugated to succinic acid (2 equiv) in the presence of DMAP (1 equiv) to make PTX-succinate, which was then reacted with EDC (1 equiv) and sNHS (1 equiv) to synthesize PTX-sNHS.

FIG. 5 shows efficacy of intratumoral paclitaxel NHS as compared to intratumoral paclitaxel.

FIG. 6 illustrates the synthesis of Dox-PL-NHS, a photocleavable doxorubicin-sulfoNHS conjugate. This conjugate uses sulfo-NHS to anchor the chemotherapeutic doxorubicin to tissues. Doxorubicin is released through the action of light, with cleaves the nitrobenzyl group to release doxorubicin.

FIG. 7 shows the synthesis of Dox-NHS, a non-cleavable doxorubicin-sulfoNHS conjugate. This conjugate uses sulfo-NHS to anchor the chemotherapeutic doxorubicin to tissues. Doxorubicin cannot be released with this example molecule.

FIG. 8 illustrates the light-mediated cleavage in mouse to release doxorubicin. Dox-PL-NHS and Dox-NHS were injected intradermally into mice. Three days after intradermal injection, the mice were imaged by live animal imaging to visualize doxorubicin. Mice were submitted to irradiation with 405 nm light, which cleaves the photocleavable group (PL) to release Dox.

FIG. 9 illustrates the synthesis of erlotinib with aryl sulfone linker for sustained covalent release of chemotherapeutic erlotinib.

FIGS. 10A and 10B illustrate the 1H NMR (FIG. 10A) and 13C NMR (FIG. 10B) data for erlotinib conjugate with an aryl-sulfone linker for release of erlotinib to tissues.

FIG. 11 shows the release of erlotinib from its prodrug through cleavage of an arylsulfone linker. 100 μM erlotinib prodrug was dissolved in 20% N-Methyl-2-Pyrrolidone (NMP), 80% phosphate buffer (10 mM, pH 7.4) and incubated at 37° C. on a rotisserie-style rotator (Labquake, Barnstead International, model number M107625) for a period of eight days. Samples were taken at hour(s) 0, 2, 5, 10, 18, 30, 48, 72, and 192 and ran through HPLC (Agilent 1290 Infinity Series.) The column was an Agilent Eclipse C18 column (2.1×50 mm; catalog number B16306) heated to 40° C. Mobile phase consisted of a gradient of 95% water, 5% methanol and 100% methanol. Free erlotinib was measured at a wavelength of 346 nm. The intact erlotinib prodrug was measured at 336 nm.

FIG. 12 is a plot showing the percent of the initial injection dose (% ID) maintained one day and one week following injection of Cy7-maleimide alone, Cy7-maleimide plus tris(2-carboxyethyl)phosphine (TCEP), and Cy7-maleimide plus mercaptoethanol (MCE).

FIG. 13 illustrates the intratumoral anchoring of Cy7-maleimide after one week.

FIGS. 14A-14D show the impact of intratumoral anchoring and local tumor release of a chemotherapeutic agent (aldoxorubicin) functionalized with a maleimide tissue binding moiety. FIG. 14A plots the percent change in tumor volume as a function of days after injection for mice treated with saline injection alone (control), intravenous injection of aldoxorubicin, and intratumoral injection of aldoxorubicin. FIG. 14B is a plot showing the probability of survival for mice treated with saline injection alone (control), intravenous injection of aldoxorubicin, and intratumoral injection of aldoxorubicin. FIG. 14C is a plot comparing tumor volume 7 days after treatment with saline injection alone (control), treatment with intravenous injection of aldoxorubicin, and treatment with intratumoral injection of aldoxorubicin. FIG. 14D is a plot comparing tumor volume 14 days after treatment with saline injection alone (control), treatment with intravenous injection of aldoxorubicin, and treatment with intratumoral injection of aldoxorubicin.

FIGS. 15A-15B schematically illustrate the Tissue Reactive Anchoring of Pharmaceutics (TRAP) technology. As shown generally in FIG. 15A, an injectable prodrug consists of a drug (FIG. 15A, star) conjugated to an anchoring motif (FIG. 15A, X) through a linker, such as a cleavable linker. When injected into tissues, the conjugate reacts with tissue extracellular matrix, anchoring the drug to the tissues. Over time, the linker connecting the drug to matrix slowly dissolves releasing drug to the tissue. FIG. 15B illustrates the TRAP strategy for an example conjugate (paclitaxel NHS).

FIGS. 16A-16G illustrate the In vivo anchoring and distribution of TRAPS within ectopic pancreatic tumors. FIG. 16A schematically illustrates the experimental methodology used to assess formation and retention of intratumoral AF647 depots. FIG. 16B shows representative IVIS images of tumor fluorescence after intratumoral injection of intact AF647-NHS or AF647-NHS inactivated by hydrolysis. FIG. 16C shows the quantification of tumor fluorescence after intratumoral injection of intact AF647-NHS or AF647-NHS inactivated by hydrolysis. FIG. 16D shows the isosurface visualization of AF647 signal in extracted cleared tumors 72 hours post intratumoral injection of intact or inactivated AF647-NHS. FIG. 16E shows the volumetric quantification of reacted AF647 visualized within the mouse tumors. FIG. 16F schematically illustrates the experimental approach to testing TRAP anchoring in excised human pancreatic tumor. FIG. 16G shows the visualization of AF647 signal in human pancreatic tumors injected with AF647-NHS. Scale bar: 200 μm, *p<0.05, by Student's t-test.

FIGS. 17A-17B illustrate the synthesis and in vitro hydrolysis of TRAP paclitaxel. FIG. 17A illustrates a synthetic methodology in which Paclitaxel (1 eqiv), succinic anhydride (2 eqiv) and DMAP (1 eqiv) were reacted in anhydrous DCM to afford paclitaxel succinic acid. Purified paclitaxel succinic acid (1 eqiv), EDC (1 eqiv) and sNHS (1 eqiv) were then reacted in anhydrous DCM to afford paclitaxel sNHS. FIG. 17B shows the kinetics of paclitaxel succinate 2 hydrolysis at pH 6.5 and 7.4 (solid line) and release of free paclitaxel (dashed lines) over time.

FIGS. 18A-18C illustrate that TRAP paclitaxel provides long-term apoptosis induction in ectopic pancreatic tumors. FIG. 18A schematically illustrates the experimental methodology used to assess the efficacy of TRAP paclitaxel for the treatment of ectopic pancreatic tumors. FIG. 18B shows the quantification of cleaved caspase 3 stained tumor sections to determine extent of apoptosis. FIG. 18C shows representative images of scanned slides showing the whole tissue section and 10× magnification. Scale bar: 200 μm. *p<0.05, **p<0.01, ***p<0.005 by Student's t-test with Holm Sidak correction for multiple comparisons.

FIGS. 19A-19F illustrate the therapeutic efficacy of TRAP paclitaxel following an intratumoral anti-cancer therapy. FIG. 19A schematically illustrates the timeline of an experiment used to evaluate TRAP in intratumoral anti-cancer therapy. FIG. 19B illustrates the excellent antitumor efficacy of TRAP paclitaxel following single intratumoral administration in an ectopic pancreatic tumor bearing mice. Inset graph showing therapeutic efficacy of two different concentrations of TRAP paclitaxel and free paclitaxel. FIG. 19C shows the tumor weights of extracted tumors. FIG. 19D is a plot showing animal weights throughout the treatment duration. FIG. 19E includes representative images of excised tumors. FIG. 19F shows histological sections of organs and tumors. Scale bar: 200 μm. *p<0.05, ****p<0.0001 by Student's t-test.

### DETAILED DESCRIPTION

### Definitions

In order that the present invention may be more readily understood, certain term are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited. Ranges provided herein are understood to be shorthand for all of the values within the range. For example, a range of 1 to 50 is understood to include any number, combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O))₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O))₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O))₂NH₂.

As used herein, the term “Cₙ₋ₘ alkylaminosulfonylamino” refers to a group of formula —NHS(O))₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O))₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O))₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O)), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “direct bond” or “bond” refers to a single, double or triple bond between two groups. In certain embodiments, a “direct bond” refers to a single bond between two groups

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

Compositions, Systems, and Methods

Disclosed herein are compositions, systems, and methods for the delivery (e.g., local and sustained delivery) of an active agent to a target tissue. The strategies involve the anchoring of an active agent (e.g., a therapeutic, diagnostic, or prophylactic agent) within a target tissue (where it can be later released and/or function as desired). Target tissue can be, for example, a tumor, an area of ischemia (heart attack, stroke), an area of local infection, an area of immunological organ injection, an area of inflammation, or other localized disease.

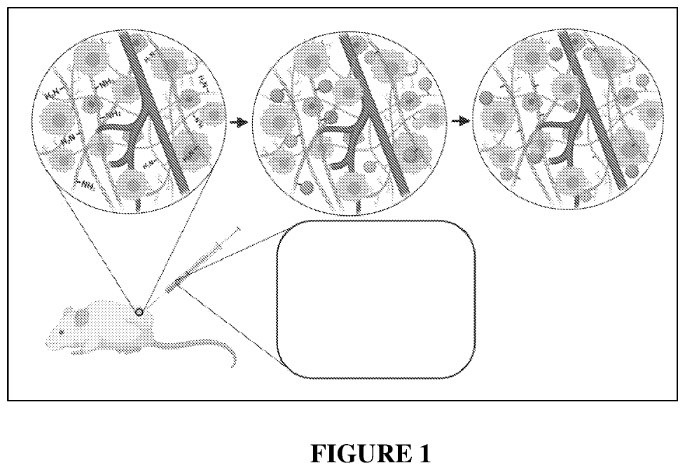

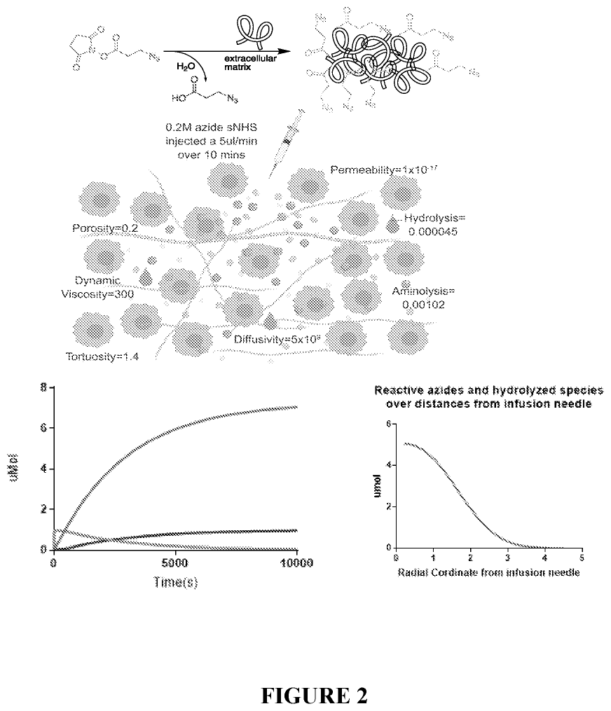

FIG. 1 schematically illustrate methods for drug delivery to a target tissue. As shown in FIG. 1, a target tissue is exposed to an anchorable active agent. The anchorable active agent includes an active agent conjugated to a tissue binding moiety, optionally by way of a linking group. In this example, the tissue binding moiety includes an amine-reactive NHS (N-hydroxysuccinimide) ester or sulfo-NHS ester. The amine-reactive NHS (N-hydroxysuccinimide) ester or sulfo-NHS ester reacts with amines present in extracellular matrix (ECM) proteins to form a covalent bond. However, it will be understood that other functional groups capable of chemically reacting with a functional group in a peptide to form a covalent bond can also function as a tissue binding moiety. As shown in FIG. 1, following treatment with the anchorable active agent, ECM proteins within the target tissue display an active agent (e.g., a therapeutic, diagnostic, or prophylactic agent) covalently tethered to the ECM proteins.

In certain examples, the tissue binding moiety can be conjugated to active agent (e.g., a therapeutic, diagnostic, or prophylactic agent) through a cleavable bivalent linker. In these embodiments, the cleavable bivalent linker can subsequently be cleaved (e.g., via hydrolysis, enzymatic reaction, exposure to an external stimulus, etc.) releasing the active agent into the target tissue over time. Based on the nature of the cleavable linker, the cleavage rate (and by extension the drug delivery profile of the active agent) can be tuned to provide controlled delivery of the active agent.

In other examples, the tissue binding moiety can be conjugated to active agent directly (i.e., the tissue binding moiety can be directly bound to the active agent) or through a non-cleavable bivalent linker. In these embodiments, the active agent can be permanently tethered to the target tissue where it can exhibit its desired activity.

Accordingly, provided herein are methods for delivering an active agent to a target tissue. These methods can comprise contacting the target tissue with a compound defined by Formula I

X-L¹-A Formula I

wherein X represents a tissue binding moiety; L¹ is absent, or represents a linking group; and A represents an active agent. Contacting the target tissue with the compound can comprise injecting or infusing a pharmaceutical composition comprising the compound into the target tissue

The target tissue can comprise any tissue in a subject which might benefit (e.g., therapeutically, prophylactically, or diagnostically) from the local delivery of an active agent. In certain embodiments, the target tissue can comprise a solid tumor.

In certain cases, the tissue can comprise tissue associated with a local cancer (e.g., pancreatic cancer, glioblastoma, breast cancer, or hepacellular carcinoma), or tissue associated with a peritoneal cancer (e.g., a sarcoma, ovarian cancer, or mesothelioma). In certain cases, the tissue can comprise a tissue associated with a local infection (e.g., an implant-associated infection, osteomyelitis). In certain cases, the tissue can comprise a transplanted tissue (e.g., an organ transplant). In certain cases, the tissue can comprise a blood contacting surface (e.g., a segment of vasculature (e.g., to prevent restenosis or thrombosis, for example, following implantation of a stent). In certain cases, the tissue can comprise a wound (e.g., to improve wound healing and regeneration). In certain cases, the tissue can comprise a ischemic sites (e.g. cardiac ischemia or peripheral artery disease). In certain cases, the tissue can comprise an ulcerated wound (e.g. diabetic ulcer). In certain cases, the tissue can comprise a site of inflammation (e.g. arthritis).

Also provided are methods of maintaining or reducing the size of a tumor in a subject in need thereof. These methods can comprise injecting or infusing into the tumor a compound defined by Formula I

X-L¹-A Formula I

wherein X represents a tissue binding moiety; L¹ is absent, or represents a linking group; and A represents an anti-cancer agent.

Also provided are methods of treating a tumor in a subject that comprise surgically resecting the tumor or a portion thereof; contacting tissue surrounding the resected tumor with a compound defined by Formula I

X-L¹-A Formula I

wherein X represents a tissue binding moiety; L¹ is absent, or represents a linking group; and A represents an anti-cancer agent.

Also provided are methods of treating a wound in a subject that comprise contacting tissue surrounding the wound with a compound defined by Formula I

X-L¹-A Formula I

wherein X represents a tissue binding moiety; L¹ is absent, or represents a linking group; and A represents a drug that promotes wound healing or vascularization.

Also provided are methods of treating or preventing an infection in a subject that comprise contacting tissue surrounding the site of infection or tissue at a site at risk for infection with a compound defined by Formula I

X-L¹-A Formula I

wherein X represents a tissue binding moiety; L¹ is absent, or represents a linking group; and A represents a drug that treats or prevents infection, such as an antibiotic.

Also provided are methods of treating or preventing immunological rejection. For example, provided herein are methods of treating or preventing graft/implant rejection in a subject that contacting grafted/implanted tissue, tissue surrounding grafted/implanted tissue, and/or tissue at the site where tissue will be grafted/implanted with a compound defined by Formula I

X-L¹-A Formula I

wherein X represents a tissue binding moiety; L¹ is absent, or represents a linking group; and A represents a drug that treats or prevents immunological/transplant rejection, such as an immunosuppressant.

Also provided are methods of treating or preventing an inflammation in a subject that comprise contacting tissue surrounding the site of inflammation or tissue at a site at risk for inflammation with a compound defined by Formula I

X-L¹-A Formula I

wherein X represents a tissue binding moiety; L¹ is absent, or represents a linking group; and A represents an anti-inflammatory drug.

Also provided are methods of treating or preventing restenosis in a subject that comprise contacting tissue surrounding a stent or tissue at the site where a stent will be deployed with a compound defined by Formula I

X-L¹-A Formula I

wherein X represents a tissue binding moiety; L¹ is absent, or represents a linking group; and A represents a drug that treats or prevent restenosis, such as an anti-proliferative drug, an anti-inflammatory drug, and/or an anti-thrombotic drug.

Also provided are methods of treating or preventing macular degeneration in a subject that comprise contacting ocular tissue or tissue adjacent to ocular tissue with a compound defined by Formula I

X-L¹-A Formula I

wherein X represents a tissue binding moiety; L¹ is absent, or represents a linking group; and A represents a drug that reduces macular degeneration, such as an anti-angiogenesis compound.

Also provided are methods of treating or preventing thrombosis in a subject that comprise contacting tissue adjacent to a blood clot or tissue adjacent to a site at risk for blood clot formation (e.g., the site of an implant such as a stent) with a compound defined by Formula I

X-L¹-A Formula I

wherein X represents a tissue binding moiety; L¹ is absent, or represents a linking group; and A represents an anti-thrombotic drug, such as an anti-platelet drug, an anticoagulant drug, a thrombolytic drug, or any combination thereof.

Tissue Binding Moieties

The compound can include any suitable tissue binding moiety. The tissue binding moiety can be any moiety which functions to anchor the active agent in the target tissue by forming a covalent bond with the target tissue. For example, in some embodiments, the tissue binding moiety can comprise a functional group capable of chemically reacting with a functional group in a peptide (e.g., an amine group, a thiol group, a carboxylate group, or a phenol group) to form a covalent bond.

In certain embodiments, the tissue binding moiety comprises a functional group capable of chemically reacting with an amine group in a peptide (e.g., an extracellular matrix protein) to form a covalent bond, such as a hydroxysuccinimidyl (NHS) group or a sulfo-hydroxysuccinimidyl (sNHS) group. Other groups that can react with amines, including acyl chlorides, isocyanate groups, sulfonyl chloride groups, aldehyde groups, acyl azide groups, anhydrides, fluorobenzene groups, tetrafluorophenyl esters, 4-sulfotetrafluorophenyl esters, carbonates, imidoester groups, epoxides and fluorophenyl esters, and dichlorotriazines can also be used. In other embodiments, the tissue binding moiety comprises a functional group capable of chemically reacting with a thiol group in a peptide to form a covalent bond, such as a maleimide group or an iodoacetate group. Other suitable functional groups are described, for example, in Montalbetti, C.A.G.N. and Falque, V. “Amide bond formation and peptide coupling,” Tetrahedron, 2015, 61: 10827-10852, which is hereby incorporated by reference in its entirety. In other embodiments, the tissue binding moiety comprises a functional group capable of chemically reactive with an alcohol group in a peptide to form a covalent bond, such as a dichlorotriazine.

Linking Groups

When present, the linking group can be any suitable group or moiety which is at minimum bivalent, and connects the two radical moieties to which the linking group is attached in the compounds described herein. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprises one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

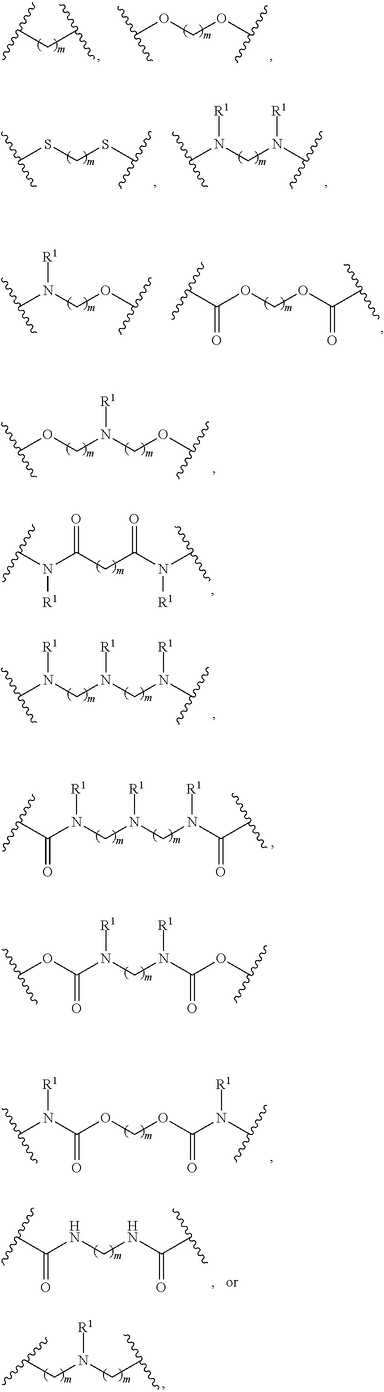

where m is an integer from 1 to 12 and R¹ is, independently for each occurrence, hydrogen, an alkyl group, an aryl group, or a heterocyclic group.

If desired, the linker can serve to modify the solubility of the compounds described herein. In some embodiments, the linker is hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

In certain embodiments, the linker can be cleavable (e.g., cleavable by hydrolysis under physiological conditions, enzymatically cleavable, or a combination thereof). Examples of cleavable linkers include a hydrolysable linker, a pH cleavage linker, an enzyme cleavable linker, or disulfide bonds that are cleaved through reduction by free thiols and other reducing agents; peptide bonds that are cleaved through the action of proteases and peptidase; nucleic acid bonds cleaved through the action of nucleases; esters that are cleaved through hydrolysis either by enzymes or through the action of water in vivo; hydrazones, acetals, ketals, oximes, imine, aminals and similar groups that are cleaved through hydrolysis in the body; photocleavable bonds that are cleaved by the exposure to a specific wavelength of light; mechano-sensitive groups that are cleaved through the application of ultrasound or a mechanical strain (e.g., a mechanical strain created by a magnetic field on a magneto-responsive gel). In other embodiments, the linker can be “click cleavable” (i.e., a click-to-release linker). Such linkers are cleaved when a click motif to which the linker is bound participates in a click reaction. Examples of click cleavable linkers (and associated click motifs) are known in the art. See, for example, Versteegen et al. Angew. Chem. Int. Ed., 2018, 57(33): 10494-10499; Versteegen et al. Angew. Chem. Int. Ed., 2013, 52(52): 14112-14116; U.S. Patent Application Publication No. 2019/0247513; and U.S. Pat. No. 10,004,810; each of which is hereby incorporated by reference in its entirety. In embodiments where an external stimulus (e.g., irradiation by light or application of a magnetic field) induces cleavage, the methods described herein can further comprise the step of applying the external stimulus to induce cleavage. Based on the nature of the cleavable linker, the cleavage rate (and by extension the drug delivery profile of the active agent) can be tuned to provide controlled delivery of the active agent.

In other embodiments, the linker can be non-cleavable. In some cases, non-cleavable linker(s) can be utilized with it is desirable that the active agent be retained (as opposed to released) once covalently tethered to the tissue. This can be the case, for example, when the active agent is an imaging agent (e.g., a contrast agent), an agent for photothermal or photodynamic therapy, or a radionuclide.

Active Agents

The term “Active Agent”, as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder.

The active agent can be a small molecule, or a biologic. A biologic is a medicinal product manufactured in, extracted from, or semi-synthesized from biological sources which is different from chemically synthesized pharmaceuticals. In some embodiments, biologics used as the active agent can include, for example, antibodies, blood components, allergenics, gene therapies, and recombinant therapeutic proteins. Biologics can comprise, for example, sugars, proteins, or nucleic acids, and they can be isolated from natural sources such as human, animal, or microorganism.

In some embodiments, the active agent can comprise an anti-cancer drug, a drug that promotes wound healing, a drug that treats or prevents infection, or a drug that promotes vascularization. For example, the active agent can comprise an anti-cancer drug, such as a chemotherapeutic or a cancer vaccine. The anti-cancer drug can include a small molecule, a peptide or polypeptide, a protein or fragment thereof (e.g., an antibody or fragment thereof), or a nucleic acid.

Exemplary anti-cancer drugs can include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine (Liposomal), Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox, Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine (Recombinant), HPV Quadrivalent Vaccine (Recombinant), Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I¹³¹ Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate).

In some embodiments, the active agent can comprise a drug that promotes wound healing or vascularization. In some embodiments, the active agent can comprise a drug that reduces ischemia, e.g., due to peripheral artery disease (PAD) or damaged myocardial tissues due to myocardial infarction. For example, the drug can comprise a protein or fragment thereof, e.g., a growth factor or angiogenic factor, such as vascular endothelial growth factor (VEGF), e.g., VEGFA, VEGFB, VEGFC, or VEGFD, and/or IGF, e.g., IGF-1, fibroblast growth factor (FGF), angiopoietin (ANG) (e.g., Ang1 or Ang2), matrix metalloproteinase (MMP), delta-like ligand 4 (DLL4), paclitaxel, or combinations thereof. Drugs that promote wound healing or vascularization are non-limiting, as the skilled artisan would be able to readily identify other drugs that promote wound healing or vascularization.

In some embodiments, the active agent can comprise an anti-proliferative drug, e.g., mycophenolate mofetil (MMF), azathioprine, sirolimus, tacrolimus, paclitaxel, biolimus A9, novolimus, myolimus, zotarolimus, everolimus, or tranilast. These anti-proliferative drugs are non-limiting, as the skilled artisan would be able to readily identify other anti-proliferative drugs.

In some embodiments, the active agent can comprise an anti-inflammatory drug, e.g., corticosteroid anti-inflammatory drugs (e.g., beclomethasone, beclometasone, budesonide, flunisolide, fluticasone propionate, triamcinolone, methylprednisolone, prednisolone, or prednisone); or non-steroidal anti-inflammatory drugs (NSAIDs) (e.g., acetylsalicylic acid, diflunisal, salsalate, choline magnesium trisalicylate, ibuprofen, dexibuprofen, naproxen, fenoprofen, ketoprofen, dexketoprofen, fluribiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, H-harpaide, or lysine clonixinate). These anti-inflammatory drugs are non-limiting, as the skilled artisan would be able to readily identify other anti-inflammatory drugs.

In some embodiments, the active agent can comprise a drug that prevents or reduces transplant rejection, e.g., an immunosuppressant. Exemplary immunosuppressants include calcineurin inhibitors (e.g., cyclosporine, Tacrolimus (FK506)); mammalian target of rapamycin (mTOR) inhibitors (e.g., rapamycin, also known as Sirolimus); antiproliferative agents (e.g., azathioprine, mycophenolate mofetil, mycophenolate sodium); antibodies (e.g., basiliximab, daclizumab, muromonab); corticosteroids (e.g., prednisone). These drugs that prevent or reduce transplant rejection are non-limiting, as the skilled artisan would be able to readily identify other drugs that prevent or reduce transplant rejection.

In some embodiments, the active agent can comprise an anti-thrombotic drug, e.g., an anti-platelet drug, an anticoagulant drug, or a thrombolytic drug.

Exemplary anti-platelet drugs include an irreversible cyclooxygenase inhibitor (e.g., aspirin or triflusal); an adenosine diphosphate (ADP) receptor inhibitor (e.g., ticlopidine, clopidogrel, prasugrel, or tricagrelor); a phosphodiesterase inhibitor (e.g., cilostazol); a glycoprotein IIB/IIIA inhibitor (e.g., abciximab, eptifibatide, or tirofiban); an adenosine reuptake inhibitor (e.g., dipyridamole); or a thromboxane inhibitor (e.g., thromboxane synthase inhibitor, a thromboxane receptor inhibitor, such as terutroban). These anti-platelet drugs are non-limiting, as the skilled artisan would be able to readily identify other anti-platelet drugs.

Exemplary anticoagulant drugs include coumarins (e.g., warfarin, acenocoumarol, phenprocoumon, atromentin, brodifacoum, or phenindione); heparin and derivatives thereof (e.g., heparin, low molecular weight heparin, fondaparinux, or idraparinux); factor Xa inhibitors (e.g., rivaroxaban, apixaban, edoxaban, betrixaban, darexaban, letaxaban, or eribaxaban); thrombin inhibitors (e.g., hirudin, lepirudin, bivalirudin, argatroban, or dabigatran); antithrombin protein; batroxobin; hementin; and thrombomodulin. These anticoagulant drugs are non-limiting, as the skilled artisan would be able to readily identify other anticoagulant drugs.

Exemplary thrombolytic drugs include tissue plasminogen activator (t-PA) (e.g., alteplase, reteplase, or tenecteplase); anistreplase; streptokinase; or urokinase.

In other embodiments, the active agent can comprise a drug that prevents restenosis, e.g., an anti-proliferative drug, an anti-inflammatory drug, or an anti-thrombotic drug. Exemplary anti-proliferative drugs, anti-inflammatory drugs, and anti-thrombotic drugs are described herein.

In some embodiments, the active agent can comprise a drug that treats or prevents infection, e.g., an antibiotic. Suitable antibiotics include, but are not limited to, beta-lactam antibiotics (e.g., penicillins, cephalosporins, carbapenems), polymyxins, rifamycins, lipiarmycins, quinolones, sulfonamides, macrolides lincosamides, tetracyclines, aminoglycosides, cyclic lipopeptides (e.g., daptomycin), glycylcyclines (e.g., tigecycline), oxazonidinones (e.g., linezolid), and lipiarmycines (e.g., fidazomicin). For example, antibiotics include erythromycin, clindamycin, gentamycin, tetracycline, meclocycline, (sodium) sulfacetamide, benzoyl peroxide, and azelaic acid. Suitable penicillins include amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, and ticarcillin. Exemplary cephalosporins include cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cfcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, ceftazidime, cefclidine, cefepime, ceflurprenam, cefoselis, cefozopran, cefpirome, cequinome, ceftobiprole, ceftaroline, cefaclomezine, cefaloram, cefaparole, cefcanel, cefedrlor, cefempidone, cefetrizole, cefivitril, cefmatilen, cefmepidium, cefovecin, cefoxazole, cefrotil, cefsumide, cefuracetime, and ceftioxide. Monobactams include aztreonam. Suitable carbapenems include imipenem/cilastatin, doripenem, meropenem, and ertapenem. Exemplary macrolides include azithromycin, erythromycin, larithromycin, dirithromycin, roxithromycin, and telithromycin. Lincosamides include clindamycin and lincomycin. Exemplary streptogramins include pristinamycin and quinupristin/dalfopristin. Suitable aminoglycoside antibiotics include amikacin, gentamycin, kanamycin, neomycin, netilmicin, paromomycin, streptomycin, and tobramycin. Exemplary quinolones include flumequine, nalidixic acid, oxolinic acid, piromidic acid, pipemidic acid, rosoxacin, ciprofloxacin, enoxacin, lomefloxacin, nadifloxacin, norfloxacin, ofoxacin, pefloxacin, rufloxacin, balofloxacin, gatifloxacin, repafloxacin, levofloxacin, moxifloxacin, pazufloxacin, sparfloxacin, temafloxacin, tosufloxacin, besifloxacin, clinafoxacin, gemifloxacin, sitafloxacin, trovafloxacin, and prulifloxacin. Suitable sulfonamides include sulfamethizole, sulfamethoxazole, and trimethoprim-sulfamethoxazone. Exemplary tetracyclines include demeclocycline, doxycycline, minocycline, oxytetracycline, tetracycline, and tigecycline. Other antibiotics include chloramphenicol, metronidazole, tinidazole, nitrofurantoin, vancomycin, teicoplanin, telavancin, linezolid, cycloserine, rifampin, rifabutin, rifapentin, bacitracin, polymyxin B, viomycin, and capreomycin. The skilled artisan could readily identify other antibiotics useful in the devices and methods described herein.

In some embodiments, the active agent can comprise a drug that reduces macular degeneration. One common current treatment for macular degeneration involves the injection of anti-angiogenesis compounds intraocularly (Lucentis, Eylea). The repeated intraocular injections are sometimes poorly tolerated by patients, leading to low patient compliance. As described herein, the ability to noninvasively refill drug depots for macular degeneration significantly improves patient compliance and patient tolerance of disease, e.g., macular degeneration, treatment. Controlled, repeated release made possible by the methods described herein allows for fewer drug dosings and improved patient comfort.

In some embodiments, the active agent can comprise a drug that prevents immunological rejection. Prior to the invention described herein, to prevent immunological rejection of cells, tissues or whole organs, patients required lifelong therapy of systemic anti-rejection drugs that cause significant side effects and deplete the immune system, leaving patients at greater risk for infection and other complications. The ability to locally release anti-rejection drugs and to repeatedly load compound allows for more local anti-rejection therapy with fewer systemic side effects, improved tolerability and better efficacy.

In some embodiments, the active agent can comprise a drug that prevents thrombosis. Some vascular devices such as vascular grafts and coated stents suffer from thrombosis, in which the body mounts a thrombin-mediated response to the devices. Anti-thrombotic drugs, released from these devices, allows for temporary inhibition of the thrombosis process, but the devices have limited drugs and cannot prevent thrombosis once the drug supply is exhausted. Since these devices are implanted for long periods of time (potentially for the entire lifetime of the patient), temporary thrombosis inhibition is not sufficient. The ability to repeatedly and locally administer anti-thrombotic drugs and release the drug significantly improves clinical outcomes and allows for long-term thrombosis inhibition.

In some embodiments, the active agent can comprise a drug that treats inflammation. Chronic inflammation is characterized by persistent inflammation due to non-degradable pathogens, viral infections, or autoimmune reactions and can last years and lead to tissue destruction, fibrosis, and necrosis. In some cases, inflammation is a local disease, but clinical interventions are almost always systemic. Anti-inflammatory drugs given systemically have significant side-effects including gastrointestinal problems, cardiotoxicity, high blood pressure and kidney damage, allergic reactions, and possibly increased risk of infection. The ability to repeatedly and locally release anti-inflammatory drugs such as NSAIDs and COX-2 inhibitors could reduce these side effects. These methods can provide the ability to deliver long term and local anti-inflammatory care while avoiding systemic side effects.

Other suitable active agents include, for example, immunotherapeutics/immunoadjuvants such as checkpoint inhibitors and STING agonists and agonists for toll-like receptors. Examples include STING ligands (e.g., natural cyclic dinucleotides, cAIMP dinucleotide, fluorine-containing cyclic dinulcoetides, phosphorothioate-containing cyclic dinucleotides, DMXAA); TLR2 ligands; TLR3 ligands (e.g., poly(I:C)); TLR4 ligands (e.g., lipopolysaccharides, monophosphoryl lipid A, CRX-527); TLR5 ligands; TLR7/8 ligands (e.g., gardiquimod, imiquimod, loxoribine, resiquimod, imidazoquinolines, adenine base analogs, benzoazepine analogs); TLR9 ligands (e.g., natural CpG ODNs, phosphorothioate CpG ODNs); TLR13 ligands (e.g., rRNA-derived ODNs); and NOD ligands (e.g., iE-DAP, meso-lanthionine tripeptide, D-gamma-Glu-mDAP, L-Ala-gamm-D-Glu-mDAP).

By way of example, representative active agents include doxorubicin, paclitaxel, gemcitabine, topotecan, tacrolimus, mycophenolic acid, rapamycin, tesiquimod, erlotinib, DMXAA, CdN, temozolomide, and docetaxel.

By way of non-limiting illustration, examples of certain embodiments of the present disclosure are given below.

### EXAMPLES

### Example 1: Anchoring of Active Agents Via NHS Ester Reactions and Subsequent Delivery of the Active Agents

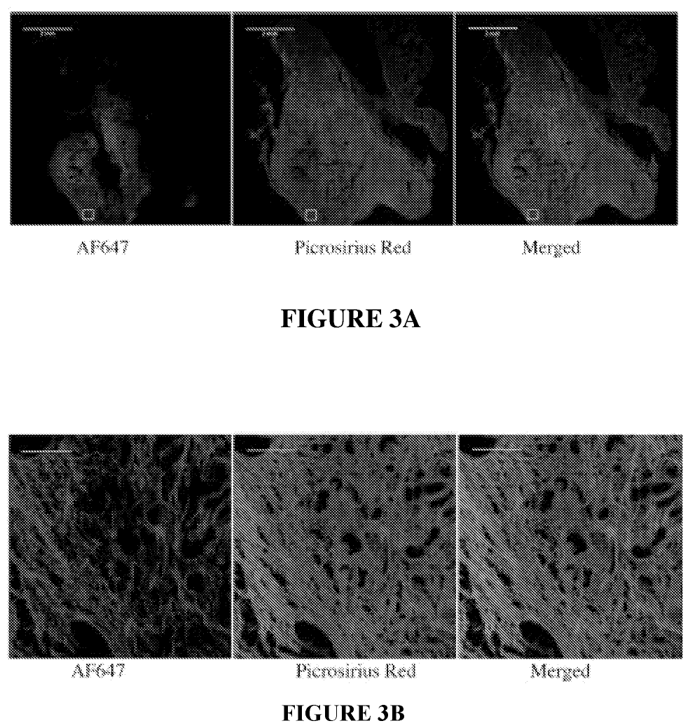

In an initial study, a model anchorable drug compound was prepared. The model compound included a reactive NHS covalently tethered to a fluorophore. Pancreatic KPC 4662 tumors were injected intratumorally with the model compound (the fluorescent NHS ester). After 24 h, the tumors were excised, fixed, sectioned, and stained with picrosirius red (which stains extracellular matrix proteins). The tumors were then evaluated via fluorescence microscopy to evaluate the co-localization of the model anchorable drug compound with extracellular matrix proteins. As shown in FIGS. 3A and 3B, the model anchorable drug compound appears co-localized with extracellular matrix proteins in the tumor, suggesting that the model anchorable drug compound covalently bonds with extracellular matrix proteins in the tumor (tethering the fluorophore in these locations). This model validated the concept that an active agent (e.g., a therapeutic agent, prophylactic agent, and/or diagnostic agent) could be tethered to a target tissue (e.g., for later release and/or interrogation).

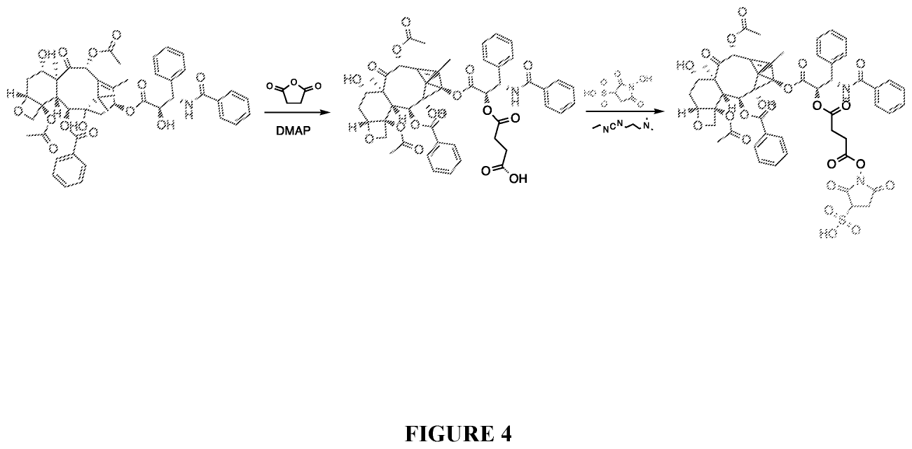

In a proof-of-principle study, an anchorable drug compound comprising paclitaxel tethered to a reactive NETS-ester. The synthetic strategy used to prepare this anchorable paclitaxel compound is shown in FIG. 4. Briefly, paclitaxel (PTX) was conjugated to succinic acid (2 equiv.) in the presence of DMAP (1 equiv.) to afford a PTX-succinate. The PTX-succinate was then reacted with EDC (1 equiv.) and sNHS (1 equiv.) to provide PTX-sNHS.

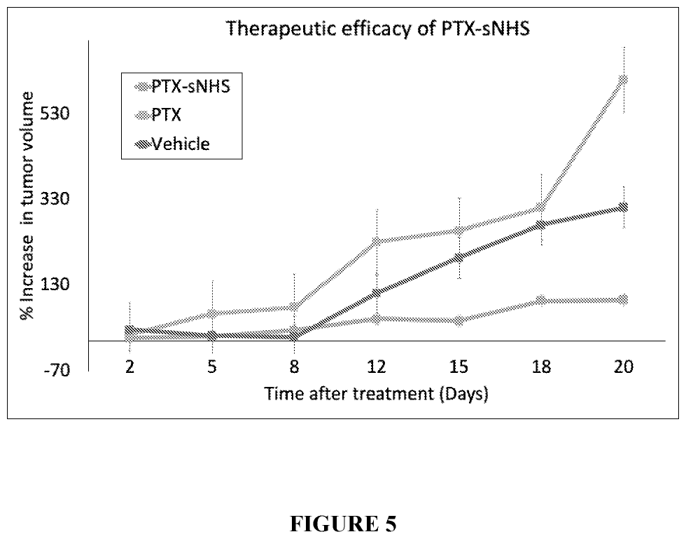

The anchorable paclitaxel compound was then administered by intratumoral injection. FIG. 5 is a plot showing the percent increase in tumor volume over 20 days following intratumoral injection of PTX-sNHS. As shown in FIG. 5, a much smaller increase in tumor volume was observed following intratumoral injection of PTX-sNHS as compared to intratumoral paclitaxel (or a vehicle control).

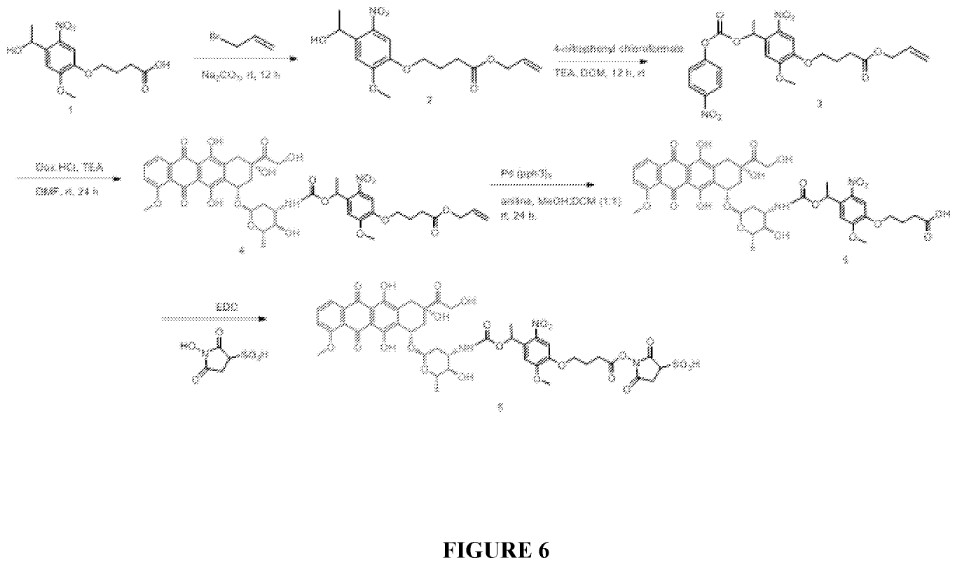

In a second proof-of-principle study, an anchorable drug compound comprising doxorubicin tethered to a reactive NETS-ester via a photocleavable linker. FIG. 6 illustrates the synthetic strategy used to prepare of Dox-PL-NHS, a photocleavable doxorubicin-sulfoNHS conjugate. This conjugate uses sulfo-NHS to anchor the chemotherapeutic doxorubicin to tissues. Doxorubicin can then be released through the action of light, with cleaves the nitrobenzyl group to release doxorubicin.

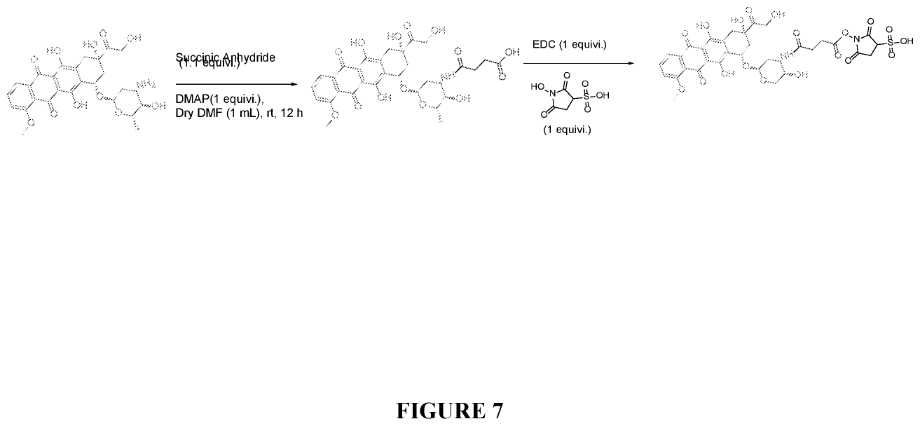

As shown in FIG. 7, a non-cleavable doxorubicin-sulfoNHS conjugate (Dox-NHS) was also prepared. Dox-NHS uses sulfo-NHS to anchor the chemotherapeutic doxorubicin to tissues. However, doxorubicin cannot be released with this molecule as the linker is non-cleavable.

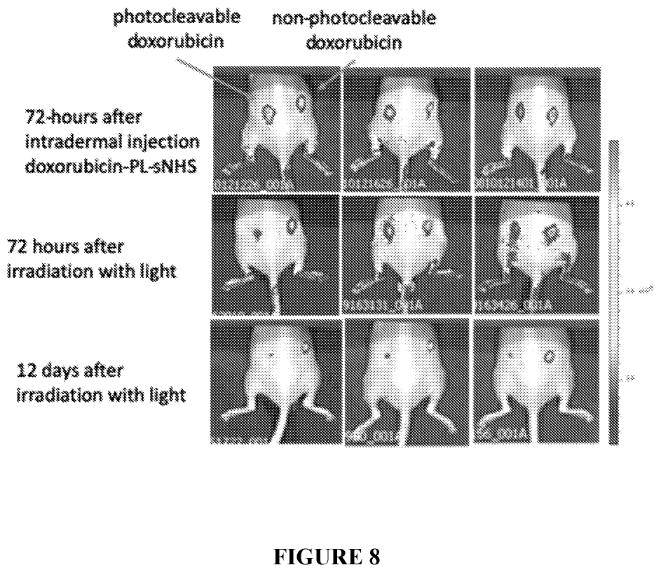

FIG. 8 shows an in vivo study evaluating the light-mediated cleavage of Dox-PL-NHS. As shown in FIG. 8, Dox-PL-NHS and Dox-NHS were injected intradermally into mice. Three days after intradermal injection, the mice were imaged by live animal imaging to visualize doxorubicin. Mice were submitted to irradiation with 405 nm light, which cleaves the photocleavable group (PL) to release Dox. As shown in FIG. 8, irradiation with light stimulated the release of Dox from the depot formed from Dox-PL-NHS. However, release was not observed from the depot formed from Dox-NHS.

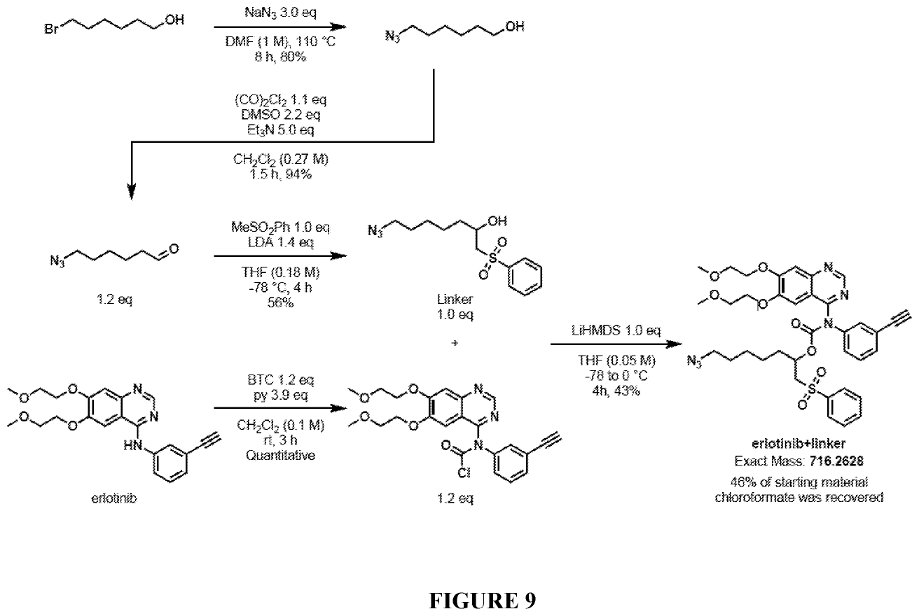

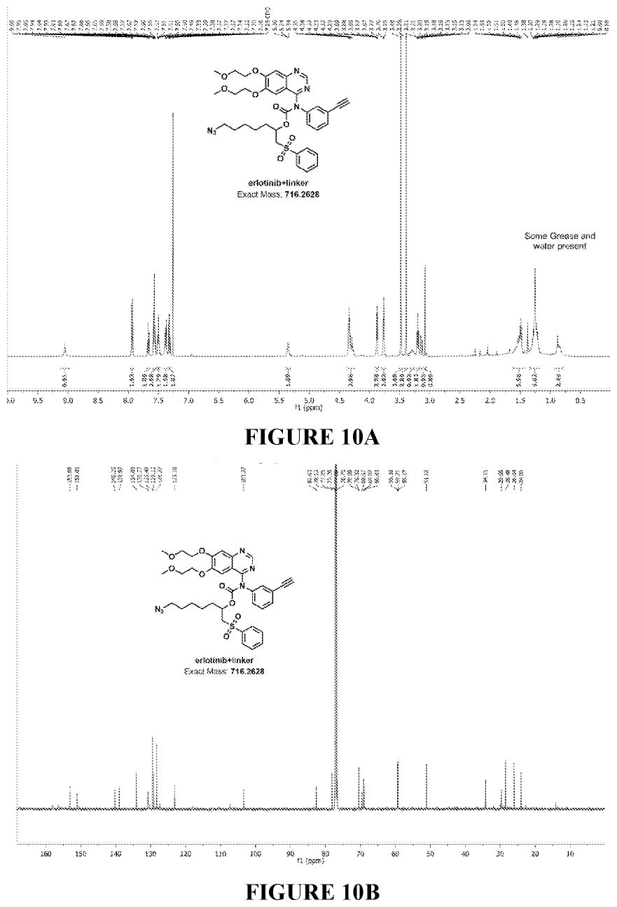

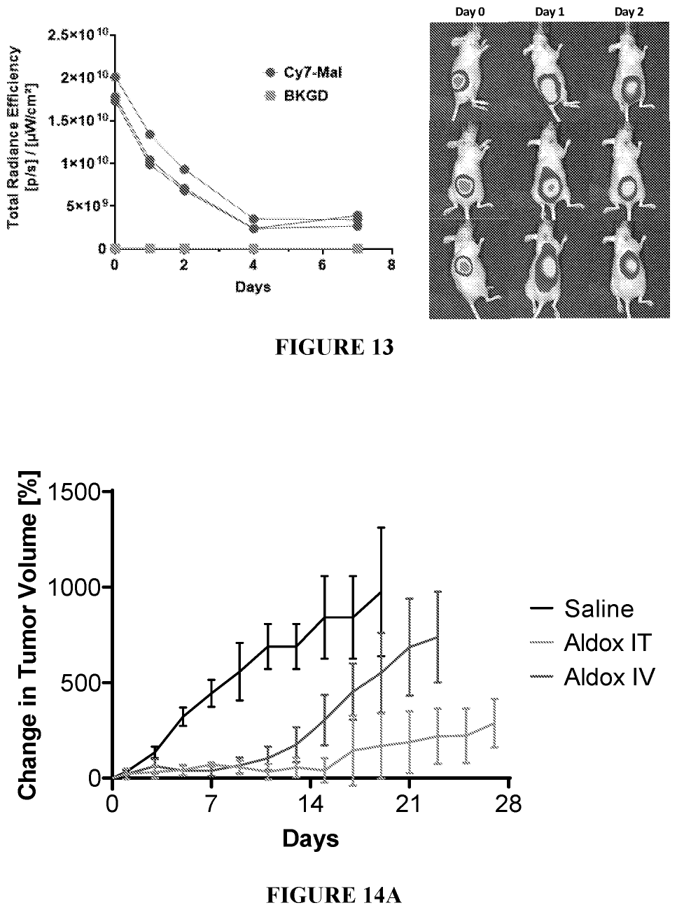

In a third proof-of-principle study, erlotinib with aryl sulfone linker was prepared for sustained covalent release of chemotherapeutic erlotinib. FIG. 9 illustrates the synthesis of erlotinib with aryl sulfone linker for sustained covalent release of chemotherapeutic erlotinib. FIGS. 10A and 10B illustrate the 1H NMR (FIG. 10A) and 13C NMR (FIG. 10B) data for erlotinib conjugate with an aryl-sulfone linker for release of erlotinib to tissues.

FIG. 11 shows the release of erlotinib from its prodrug through cleavage of an arylsulfone linker. 100 μM erlotinib prodrug was dissolved in 20% N-Methyl-2-Pyrrolidone (NMP), 80% phosphate buffer (10 mM, pH 7.4) and incubated at 37° C. on a rotisserie-style rotator (Labquake, Barnstead International, model number M107625) for a period of eight days. Samples were taken at hour(s) 0, 2, 5, 10, 18, 30, 48, 72, and 192 and ran through HPLC (Agilent 1290 Infinity Series.) The column was an Agilent Eclipse C18 column (2.1×50 mm; catalog number B16306) heated to 40° C. Mobile phase consisted of a gradient of 95% water, 5% methanol and 100% methanol. Free erlotinib was measured at a wavelength of 346 nm. The intact erlotinib prodrug was measured at 336 nm. As shown in FIG. 11, approximately 60% of the erlotinib was released over a period of approximately 200 hours.

### Example 2: Anchoring of Active Agents Via Maleimide-Thiol Reactions and Subsequent Delivery of the Active Agents

In an initial study, cyanine 7 maleimide (Cy7-maleimide) was used as a model for an anchorable drug compound containing a maleimide tissue binding moiety. Cy7-maleimide includes a reactive maleimide group covalently tethered to a fluorophore.

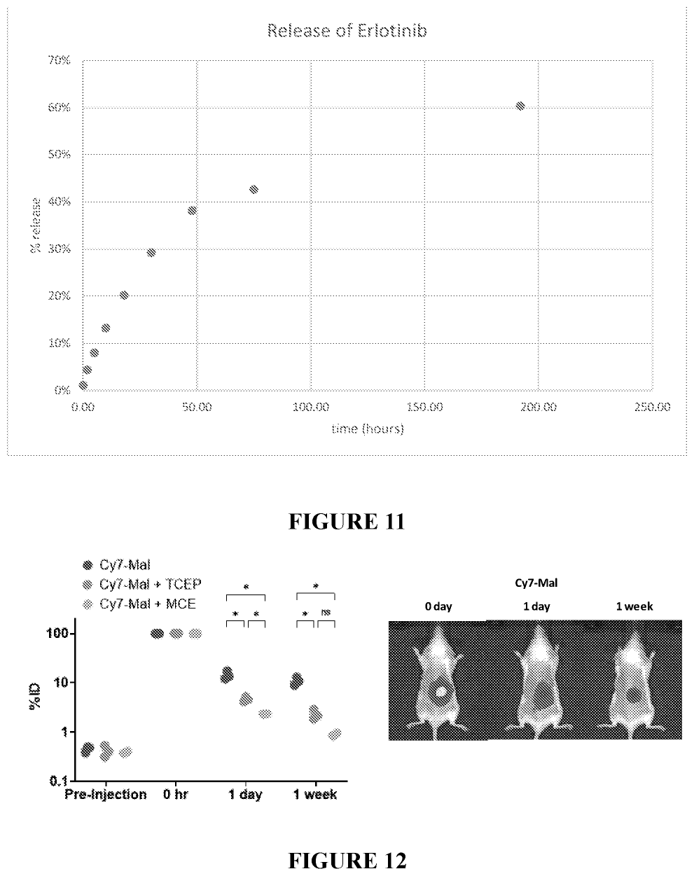

To investigate whether maleimide molecules can anchor to dermal tissue by binding to available thiol groups, CD-1 mice were injected intradermally with Cy7-maleimide (50 uL of 0.2 mM) and monitored for fluorescence over 1 week using an IVIS imager. ICG/ICG excitation and emission filters were used for all IVIS images. Cy7-maleimide plus TCEP was used to attempt to increase free thiols by breaking disulfide bonds. Cy7-maleimide plus MCE was used as control to first react with maleimides to show that anchoring of the maleimide is crucial. As shown in FIG. 12, approximately 10% of an injection dose of Cy7-maleimide was maintained after 1 week as compared to only ˜1% of the injection dose upon injection of the control (Cy7-maleimide plus MCE). No skin irritation or skin toxicity was observed.

This study suggested that the model anchorable drug compound containing a maleimide tissue binding moiety could efficiently react with thiol moieties present in proteins following injection, tethering the fluorophore to tissue at the injection site. This model validated the concept that an active agent (e.g., a therapeutic agent, prophylactic agent, and/or diagnostic agent) could be tethered to a target tissue (e.g., for later release and/or interrogation) using a maleimide tissue binding moiety as well.

To investigate whether active maleimide molecules can anchor to tissue in a tumor, nude mice were injected subcutaneously with U87 tumor cells mixed with Matrigel. Once tumor reached 100 mm³, Cy7-maleimide (50 uL of 0.2 mM) was injected intratumorally and monitored for fluorescence over 1 week using an IVIS imager. ICG/ICG excitation and emission filters were used for all IVIS images. The results are shown in FIG. 13. As shown in FIG. 13, approximately 18% of an injection dose of Cy7-maleimide dose was maintained 1 week after intratumoral injection. This validated that anchorable drug compounds containing a maleimide tissue binding moiety could efficiently react with thiol moieties present in proteins found within tumors following intratumoral injection.

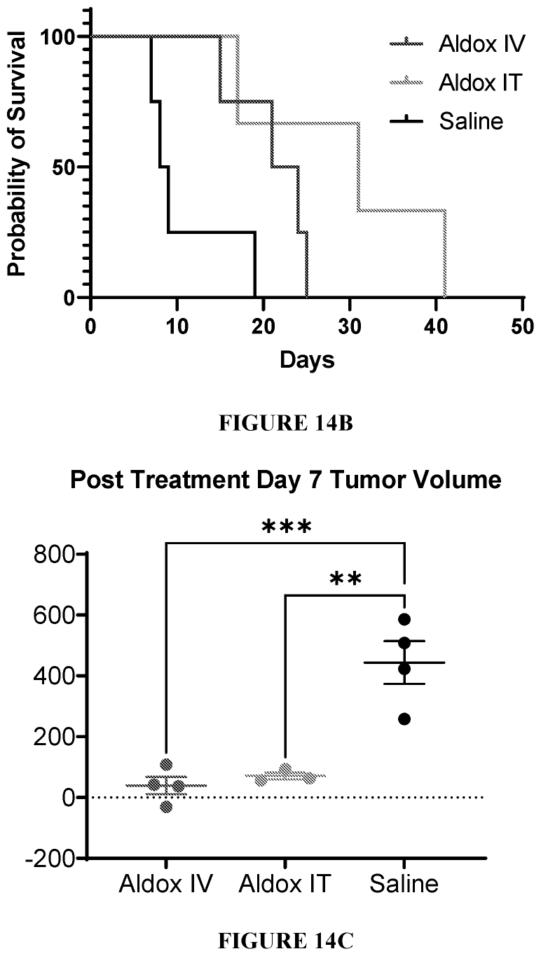

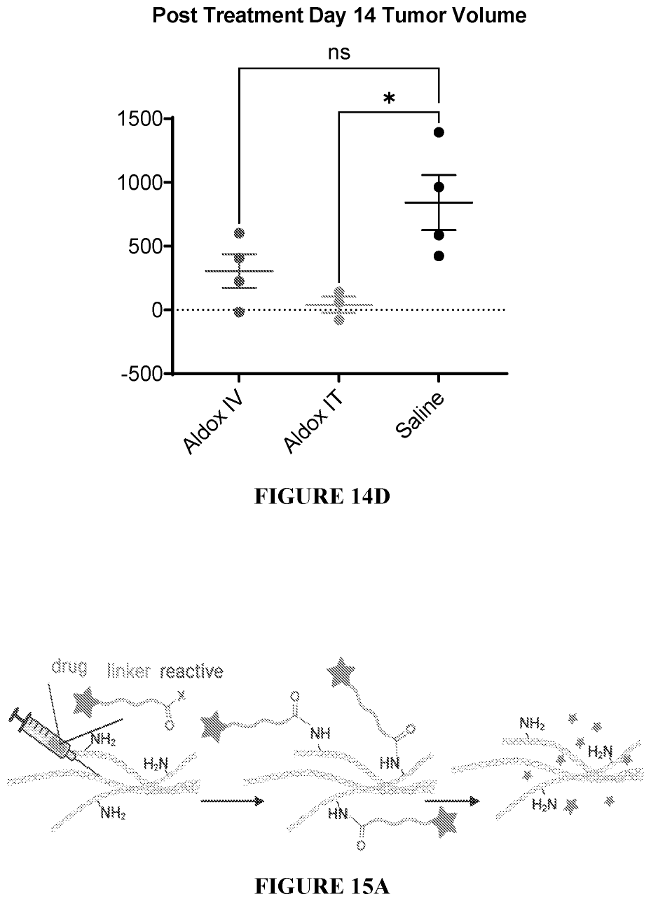

In a further proof-of-principle study, aldoxorubicin was used to validate the concept of anchoring (and subsequent local tumor release) of a chemotherapeutic agent functionalized with a maleimide tissue binding moiety. In this study, nude mice were injected subcutaneously with U87 tumor cells mixed with Matrigel. Once tumor reached 100 mm³, treatment groups were either injected with aldoxorubicin intratumorally (IT, 50 uL at a controlled rate of 5 uL/min) or intravenously (IV, 100 uL). Tumor size and weight were measured at varying intervals following injection. Mice were euthanized when tumor reached 2 cm in width in any direction. Aldoxorubicin was delivered by either IV or IT (10.5 mg/kg; 7.73 mg/kg dox eq.) and compared to saline group. As shown in FIGS. 14A-14D, aldoxorubicin administered via intratumoral injection resulted in improved tumor inhibition and survival probability as compared to both saline and aldoxorubicin administered intravenously.

### Example 3: Tissue-Reactive Drugs Enable Materials-Free Local Depots

Local, sustained drug delivery of potent therapeutics holds the promise to present constant concentrations of drugs and eliminate systemic side effects. However, introduction of viscous hydrogels or polymer-based implants for local drug delivery is highly limited in stiff tissues including desmoplastic tumors. In this example, we present a method to create material-free intratumoral drug depots by introducing Tissue-Reactive Anchoring Pharmaceuticals (TRAP) that can easily diffuse into tissue and attach locally for sustained drug release. In TRAP, potent drugs are modified with ECM-reactive groups (NHS esters) and, when injected intratumorally, quickly react with accessible amines in the ECM to create local drug depots. We demonstrate that locally injected TRAP creates dispersed, stable intratumoral depots deep within mouse and human pancreatic tumor tissues. TRAP depots based on ECM-reactive paclitaxel (TRAP paclitaxel) demonstrated better solubility than free paclitaxel and provided sustained in vitro drug release. Furthermore, TRAP paclitaxel induced higher tumoral apoptosis and sustained improved antitumor efficacy as compared to free drug. By providing continuous drug access to tumor cells, this material-free approach to create local drug delivery of potent therapeutics has the potential in a wide variety of diseases where current injectable depots fall short.

Introduction

Sustained drug release from local drug depots has the potential to overcome systemic toxicity and clearance challenges observed with systemic drug administration (1), including delivery of chemotherapy to locally advanced tumors (2), immunosuppressive agents to transplanted organs (3), antibiotics to localized infections (4), and immune regulators (5) to lymph nodes (6) and arthritic joints (7). However current local drug delivery approaches that rely on depot injection or implantation of viscous materials such as polymers (8) and hydrogels (9) struggle to penetrate stiff or inflamed tissues to deliver drugs deep into the target area (8, 10).

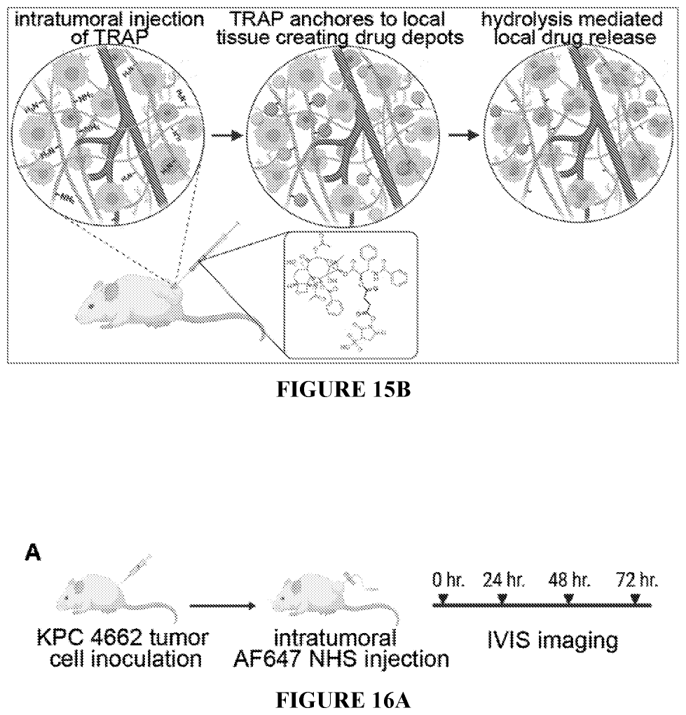

In this example, we demonstrate that local depots can be administered without the use of viscous materials. The Tissue Reactive Anchoring Pharmaceuticals (TRAP) technology creates local drug depots through chemical labelling of tissue extracellular matrix (ECM) and subsequently releases active drug locally (FIGS. 15A-15B). TRAP depots are created when drugs conjugated to ECM-reactive chemical groups are introduced into target tissues. TRAP drugs diffuse through tissue and ECM-reactive groups (NHS esters) react with accessible amines in the ECM to anchor drugs through a stable amide bond. For diseases defined by densely packed stroma, TRAP depots turn the dense tumor stroma into an asset due to the abundant presentation of primary amines in the ECM as part of lysine groups and N-termini protein (11, 12). Moreover, delivery of TRAP in non-viscous, aqueous vehicles allows radial diffusion of drug molecules in the dense stroma overcoming the primary obstacle for implant-based delivery strategies (13).

The need for improved sustained local drug delivery of potent chemotherapeutics is particularly acute in locally advanced solid tumors (14), including locally advanced pancreatic cancer (LAPC) (15). LAPC constitutes 30% of diagnosed pancreatic adenocarcinomas and comprises unresectable malignant disease without overt distant metastases (16, 17). LAPC is often treated with systemic administration of Gemcitabine and FOLFIRINOX (16, 18) but, effective management of LAPC is hampered by dense desmoplastic stroma surrounding tumor cells (19). The dense stroma is rich in fibrillar collagen and accounts for almost 70-80% of the total tumor volume. The density, reorganization and alignment of the extracellular matrix contributes to reduced blood perfusion, hypoxic tumor microenvironment and increased interstitial pressure (20) presenting a barrier for therapeutic agents administered systemically to reach and accumulate in the tumor tissue at therapeutic concentrations (21). Due to the inadequacy of systemic therapy for LAPC, attention has focused on local drug delivery solutions for control of unresectable tumors, to debulk the tumor mass, or to provide palliative care (22, 23). These strategies include locally implanted hydrogels, (24-26) polymeric matrices (27, 28), patches (29, 30) and membranes (31) inserted intratumorally or peritumorally to achieve high local drug concentration for long periods of time. Local drug delivery strategies have shown superior potential in treating locally advanced pancreatic tumors (32), but they rely on injection or implantation of materials into a stiff tissue and struggle to deliver drugs deep into the solid tumors (33). Therefore, there is an unmet need for effective local drug delivery strategies for management of locally advanced pancreatic cancer that would allow better drug penetration in the dense tumor tissue with minimum patient morbidity.

Paclitaxel is a promising candidate against a broad spectrum of solid cancers including in pancreatic cancer, showing promise in improving LAPC resectability (34). However, paclitaxel's poor solubility (35) and poor tumor penetrance (36) necessitate drug delivery approaches to improve paclitaxel presentation. Previous approaches to paclitaxel delivery, including nanoparticles (37, 38), hydrogels (39) and polymeric implants (40), have shown promise in preclinical pancreatic tumor models. Utilizing the ability of paclitaxel to effectively control pancreatic tumor growth, we sought novel approaches to deliver this potent chemotherapeutic deep within the solid tumor in a sustained manner without a viscous injections or implantation surgeries.

To effectively demonstrate the ability of TRAP depots to distribute widely throughout stiff tumors and sustainably produce anti-tumor therapy, we tested TRAP paclitaxel for the treatment of locally advanced pancreatic cancer (LAPC). In this example, we demonstrate the formation of stable intratumoral paclitaxel depots created by injecting TRAP paclitaxel into the dense pancreatic tumor. We demonstrate that TRAP can anchor molecules within mouse and human pancreatic tissues. We additionally show that TRAP-paclitaxel increases tumor apoptosis and anti-cancer efficacy as compared to free paclitaxel in a mouse model of pancreatic adenocarcinoma. This materials-free approach to sustained release of drugs at target tissue sites could be a general approach to the clinical treatment of many other diseases where localized drug presentation improves clinical therapy

Materials and Methods

Cell culture. KPC 4662 pancreatic tumor cell vials were received from the laboratory of Dr. Pylayeva-Gupta at UNC Chapel Hill at passage 9. Cells were revived and maintained in 1× high-glucose Dulbecco's modified Eagle's culture medium (DMEM) (Hyclone, cat #SH30022.01) supplemented with 10% fetal bovine serum (Gibco, cat #26140-079) and 100 U/mL penicillin/streptomycin (Fisher, cat #15-140-122) at 37° C. and 5% CO₂. To subculture, cells were washed with PBS and detached using 0.05% trypsin-ethylenediaminetetraacetic acid (EDTA) (Fisher, cat #25-300-054).

Formation and Retention of Intratumoral Depot. To evaluate the formation and retention of ECM anchored depots, AF647-NHS esters (click chemistry tools, cat #1344-1) were injected intratumorally in the ectopic pancreatic tumor bearing mice and imaging was performed at specified time intervals to visualize AF647 signal. Ectopic pancreatic tumor model was developed by subcutaneous inoculation of approximately 1*10⁶ KPC 4662 murine pancreatic tumor cells in 1:1 PBS/matrigel (Corning/cat #354234) solution on the dorsal flanks of 8-week-old female albino C57BL/6 mice (Charles River). Once the tumors were approximately 100 mm³ in volume, animals were randomly divided into two groups. Each group received intratumoral infusion of 50 μl of 0.1 mM activated AF 647 NHS ester (n=3) or hydrolyzed inactivated AF 647 (n=3) using 27 g winged catheter and a syringe pump (Harvard apparatus, cat #70-4500) at the rate of 10 μL/min for 5 minutes. All animals were imaged at 0, 4, 24, 48 and 72 hours post intratumoral infusion using IVIS Spectrum In Vivo Imaging System (IVIS). ICG BKG/ICG excitation and emission filters were used for all IVIS images presented and no image math in the Living Image software was performed. All animals were euthanized by intra-arterial perfusion of 10 ml of cold PBS under anesthesia followed by 10 ml of 10% neutral buffered formalin (Millipore Sigma, cat #34172-118) prior to tumor extraction.

To assess the ECM anchored depot formation in the human pancreatic tumors. Excised human pancreatic tumor tissue was received from a Tissue Procurement Facility at UNC Lineberger Comprehensive Cancer Center following pancreatic tumor surgery. The collected human tumor samples were cut into pieces approximately 100 mm³ volume per piece. Each piece was washed with PBS and imaged using IVIS before dye fluorophore-NHS administration. Tumor pieces (n=3) were injected with 50 μL of 0.2 mM AF647 NHS using a 27 g winged catheter and a syringe pump at the rate of 5 μL/min over 10 minutes. Control samples were untreated. Each piece was imaged immediately after dye injection and 24 hours post-injection using IVIS. AF 647 signal was further quantified to determine the depot anchoring.

Mouse and human tumors were fixed in 10% neutral buffered formalin and submitted to clearing using iDISCO protocol (47). Briefly, all samples were dehydrated using increasing concentrations of methanol (20-100%) and kept shaking at room temperature for 1 hour each. Dehydrated samples were shaken three times in dichloromethane (Acros organics, cat #348465000) for 30 min each to wash excess methanol. Finally, tissues were placed in dibenzyl ether (Sigma, cat #33630) for clearing. Clear tumors were imaged using a Lavision Ultramicroscope II and evaluated using IMARIS software version 9.7. Postprocessing using IMARIS included visualizing AF647 signal with respect to tumor autofluorescence signal (AF488) and creating isosurfaces for AF488 and AF647 signals. AF488 isosurface was made transparent to visualize red colored AF647 signal. Quantitative analysis of the created isosurfaces was performed to calculate depot volume

Synthesis and Characterization of Paclitaxel-sNHS Esters. Highly reactive ECM-anchoring PTX-sNHS conjugate was synthesized in a two-step process. Initially, PTX succinic acid was synthesized using a method described in Shan et al. (48) with slight modifications. Briefly, paclitaxel (Medkoo, cat #100690) (1 equiv.) was dissolved in dichloromethane (DCM) (Acros organics, cat #348465000) under inert conditions and reacted with succinic anhydride (TCI, cat #TCS0107)(2 equiv.) in the presence of (4-Dimethylamino) pyridine (DMAP) (Aldrich, cat #107700)(1 equiv.). The reaction was kept stirring overnight at room temperature. The crude mixture was purified using silica gel chromatography. Further, purified paclitaxel succinic acid (1 equiv.) was reacted with 1-Ethyl-3-(3-dimethylaminopropyl)carbodiimide (EDC) (Oakwood chemicals, cat #024810) (1 equiv.) and 1-Hydroxy-2,5-dioxopyrrolidine-3-sulfonic acid (sNHS) (Combi-Blocks, cat #82436-78-0) (1 equiv.) dissolved in NN-Dimethylformamide (DMF) (Acros organics, cat #348435000) under inert conditions. The reaction was kept stirring overnight at room temperature and purified using ether (Fisher, cat #AAL14030AU) precipitation. The final purified paclitaxel-sNHS conjugate was collected and further characterized using LCMS and NMR.

In Vitro PTX Release. Hydrolytic studies of PTX succinic acid were carried out at 37° C. in PBS containing 0.01% DMSO and 0.1% Tween 80 at pH 7.4 and pH 6.5. 5.33 μM of PTX-succinic acid was dissolved in a 5 ml of medium and kept continuously stirring at 150 rpm/min. 50 μL of sample was removed at predetermined time intervals of 0, 24, 48, 72 and 96 hours and replaced with 50 μL of fresh media. Removed samples were mixed with 50% of acetonitrile and analyzed using Agilent 1290 HPLC system with Agilent 2.1×50 mm C18 column and mobile phase consisting of 60% ammonium formate and 40% acetonitrile. Concentration of PTX-SA and free PTX were determined using calibration curves.

In Vivo Assessment of PTX-sNHS Induced Apoptosis. To assess the ability of intratumoral depots to release free paclitaxel and induce apoptotic tumor cell death, ectopic pancreatic tumor model was developed by subcutaneous inoculation of approximately 1*10⁶ KPC 4662 murine pancreatic tumor cells in 1:1 PBS/matrigel (Corning/cat #354234) solution on the dorsal flanks of 8 week old female albino C57BL/6 mice (Charles River). Once the tumors were approximately 100 mm³ in volume, animals were randomly divided into four groups to receive either no treatment or single intratumoral injection of vehicle (50% NMP: alfa aesar, #AA44063-K2, 50% Saline: cat #470302-026, VWR), free paclitaxel (Medkoo, cat #100690, 20 mg/kg) and paclitaxel-sNHS (n=3/group, 20 mg/kg). All animals were euthanized to collect tumors 72 hours post injection. Tumors were fixed in 10% NBF and sectioned to stain for cleaved caspase 3 and hematoxylin. Stained tumor slides were scanned and analysis of apoptosis was performed using Image J (version 2.1.0/1.53c). For quantification of apoptosis, CC-3-stained vs hematoxylin-stained areas were measured with respect to total tumor area (45, 46).

Evaluation of Antitumor Efficacy of PTX-sNHS Esters in Ectopic Model. The antitumor effect of PTX-sNHS was studied in a syngeneic subcutaneous KPC 4662 tumor model. Briefly, 5*10⁵ KPC 4662 murine pancreatic tumor cells in 1:1 PBS/matrigel (Corning/354234) suspension were injected in the dorsal flank of female C57BL/6J (Charles River) mice. Tumor growth was monitored using vernier calipers every alternate day and tumor volume was calculated using a formula (length*width*width)/2. When tumors were approximately 100 mm³ in volume, all animals were randomly divided into four groups to receive single intratumoral administration of either paclitaxel (20 mg/kg), paclitaxel-sNHS low (20 mg/kg), paclitaxel-sNHS high (50 mg/kg) or vehicle control. Each mouse was administered 50 μL of intratumoral injection of respective treatment at the rate of 5 μL/min over 10 min using 27 g winged catheter and syringe pump. Tumor growth was monitored every 2 days until one of the animals reached the experimental end point. On Day-30, all animals were euthanized and tumors and organs (heart, liver, kidneys, lungs, and spleen) were collected to evaluate local and systemic immune response. Collected tissues were immediately fixed in 10% neutral buffered formalin and submitted for further histological processing and H&E staining (Histology core at NC State College of Veterinary Medicine). Stained sections were imaged and were sent to a blinded, certified pathologist for evaluation.

Statistical analysis. All data was presented and analyzed for statistical significance using Prism software (version 9.0.1). To evaluate the statistical significance, student t test was used with Holm-Sidak correction for multiple comparison.

Results and Discussion

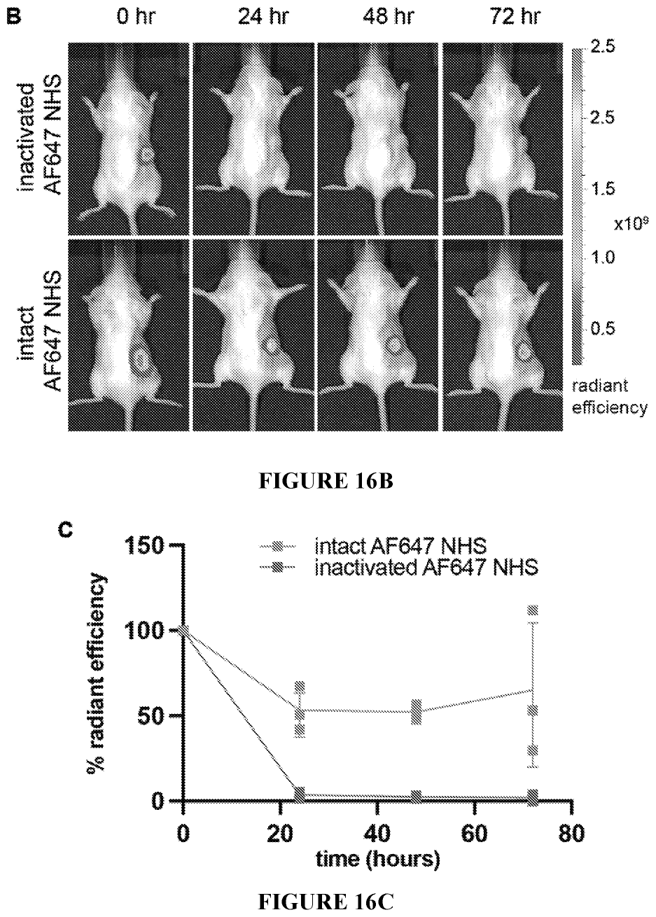

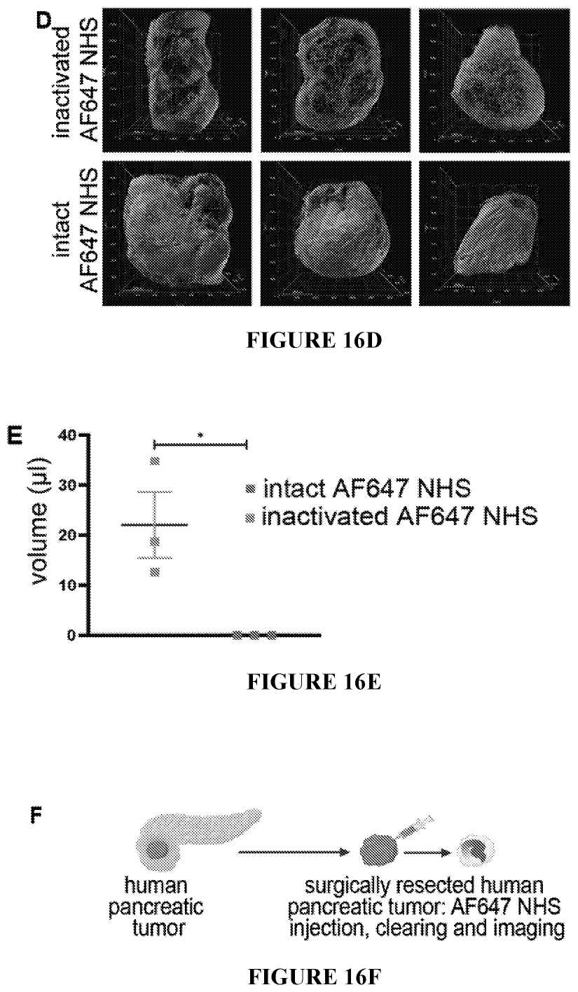

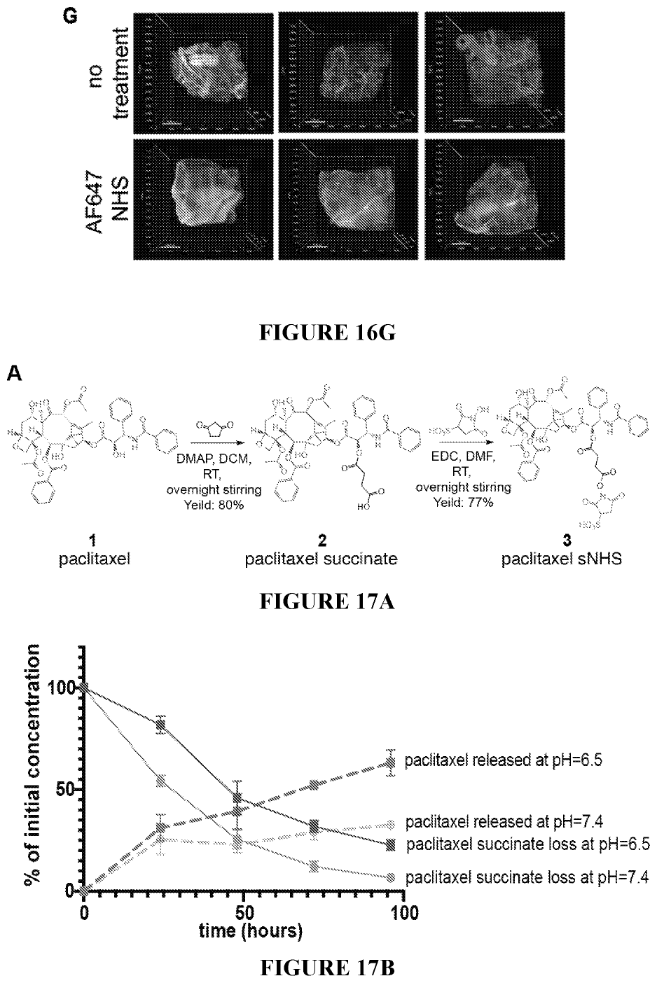

TRAP depots distribute throughout and are retained within mouse and human pancreatic tumors. Sustained release of drugs from depots benefits from widely distributed drugs that are “anchored” to their location, preventing fast drug clearance from the tissues. We hypothesized that the activated N-hydroxysuccinimide (NETS) esters of carboxylic acids, amine-reactive chemical groups commonly used to label proteins (19), could serve the purpose of anchoring molecules to tissue extracellular matrix. To study depot distribution after intratumoral introduction, we chose the KPC 4662 pancreatic tumor model due to its high stiffness and fibrosity (20), making intratumoral injections of viscous hydrogels nearly impossible. However, the collagen rich nature of these tumors allows numerous accessible amine sites for NHS reactivity. To study intratumoral depot formation, distribution and retention, we infused the NHS esters of AlexaFluor647 dye (AF647-NHS) into subcutaneous KPC4662 pancreatic tumors (FIG. 16A). As a negative control, AF647-NHS was first incubated in water to fully hydrolyze the NHS esters. Animals were imaged immediately after intratumoral injection and then daily for 72 hours using in vivo fluorescence imaging (IVIS) to visualize AF647 retention (FIG. 16B). 24 hours after intratumoral injection, the majority of the initial fluorescence signal remained in the tumors and this fraction was robustly maintained over the next 72 hours, with no further decrease in signal. In sharp contrast to the results seen with intratumoral injection of intact AF647-NHS, inactivated AF647-NHS was rapidly cleared from the tumor, with only 3% of the signal detectable at 24 hours, consistent with tumor clearance in the absence of ECM anchoring (FIG. 16C). To analyze the distribution of anchored AF647-NHS, we submitted tumors to tissue clearing and analyzed fluorescence spread by confocal imaging. 72 hours after injection, tumors were excised, cleared using the iDISCO protocol and imaged by light sheet microscopy. Confocal images were processed with IMARIS to visualize the volumetric representation of the AF647-NHS. No distinguishable fluorescence was observed in tumors infused with inactivated AF647-NHS. In sharp contrast, all tumors receiving intact AF647-NHS demonstrated distinct volumes of fluorescence signal (FIG. 16D). Some variability was noted, with some tumors demonstrating homogeneous signal throughout the entirety of the tumors, while in one tumor, a distinct localization at the tumor margins was noted. Volumetric analysis of the images showed an approximate spread volume of 22 μL, which is in good agreement with the in vivo imaging data (FIG. 16E). The decrease in observed spread volume (22 μL) vs infused volume (50 μL) may be explained by reaction and retention of NHS esters at the infusion front. Alternatively, it is possible that a fraction of injected material escapes the tumor.

Having demonstrated that TRAP AF647 efficiently labels a stiff, highly desmoplastic mouse pancreatic tumor, we tested the TRAP anchoring paradigm in human tumors. Surgically resected primary human pancreatic tumors were cut into ˜100 mm³ pieces and each piece was infused with AF647-NHS and imaged immediately after and 24 and 96 hours after infusion in the IVIS to quantify retention of fluorescent signal (FIG. 16F). Non-injected 100 mm³ pieces were used as controls. Approximately 80% of the signal was retained after 24 hours and no significant change was seen in the signal intensity up to 96 hours. The efficient retention of fluorescence in the human tumors suggests efficient anchoring and intratumoral AF647 depot formation in these tumor tissues. To evaluate the depot distribution throughout the human pancreatic tumor tissue, all pieces were submitted to iDISCO tissue clearing followed by confocal light sheet imaging. Evaluation of the AF647 signal suggests diffuse distribution of AF647 depots throughout two of the tumors and a more centralized localization of the signal for one tumor (FIG. 16G).

Taken together, these results demonstrate that TRAPs depot can be introduced into stiff pancreatic tumors of both humans and mice with wide distribution and good anchoring. The majority of injected materials is anchored within the tumors and form a stable, distributed depot throughout the tumor. A fraction of the fluorescence was lost within 24 hours, likely due to a combination of NHS ester hydrolysis and reaction with soluble factors that can drift away from the site.

Synthesis and in vitro characterization of TRAP paclitaxel. In the case of therapeutic agents that need to be released to elicit therapeutic benefit, drugs can be conjugated to reactive NHS esters through cleavable linkers in order to function within the TRAP system. In the case of paclitaxel, the ester modification to the 2′ hydroxyl has previously been described to be hydrolyzed with sustained kinetics (41). Thus, TRAP paclitaxel was synthesized from paclitaxel over two steps with 61% overall yield (FIG. 17A). First, paclitaxel was reacted with succinic anhydride in the presence of DMAP to give the 2′-O-succinyl paclitaxel 2 in 80% yield. Then, 2 was coupled to sNHS in the presence of EDC to yield paclitaxel sNHS esters in 77% yield. Purified paclitaxel succinic acid and paclitaxel sNHS esters were evaluated using LC-MS and NMR.

The hydroxyl group at the C-2′ position is necessary for paclitaxel's anticancer activities. Therefore, the succinate ester must hydrolyze to achieve desired antitumor responses (41). We measured paclitaxel release from PTX-succinate in PBS at 37° C. in both neutral pH and the slightly acidic conditions (pH˜6.5) commonly observed in the tumor microenvironments. Disappearance of the PTX succinate peak and appearance of PTX peak was quantified using HPLC. PTX-succinate demonstrated a half-life of ˜37 hours at neutral pH and ˜56 hours at pH 6.5 (FIG. 17B). After 24 hours, nearly 82% of PTX-succinate remained intact in acidic conditions, while 54% remained in the neutral condition. Interestingly, formation of free paclitaxel over time did not perfectly mirror disappearance of PTX-succinate. At neutral pH, paclitaxel concentrations reached a maximum of 32% release at 96 hours, but did not increase past this point, despite continued decrease in the concentration of paclitaxel succinate. In contrast at the more acidic pH, paclitaxel continued its accumulation in the release media, with 60% of initial dose observed at 96 hours. The observed loss of some PTX-succinate without a concomitant increase in paclitaxel suggests that at neutral pH, other side reactions were consuming paclitaxel, likely through hydrolysis of the acetyl groups on C4 or C10. The lower presence of paclitaxel in neutral conditions further suggests that accidental introduction of 2′-O modified paclitaxel as in TRAP paclitaxel into healthy surrounding tissues would lead to less paclitaxel release and, thus, lower unintended toxicity.

Taken together, these results demonstrate that TRAP paclitaxel slowly releases the parent drug at lowered pH, suggesting that TRAP is a strong candidate for intratumoral TRAP delivery.

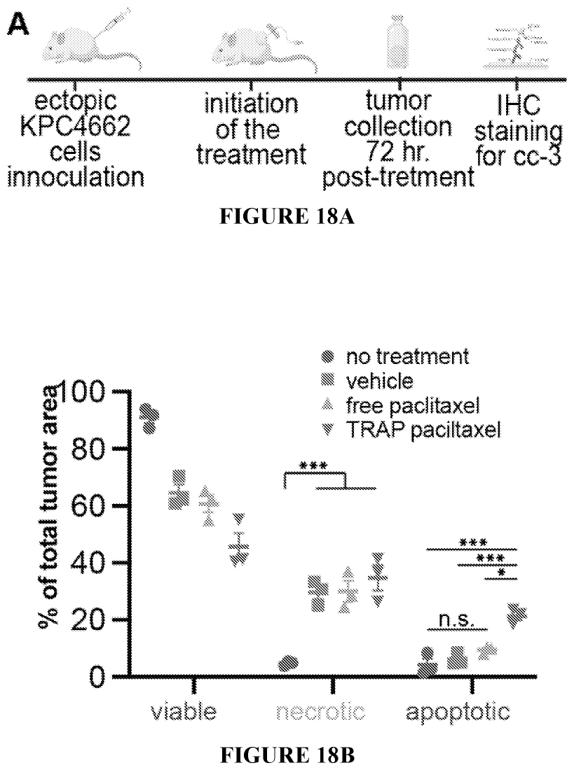

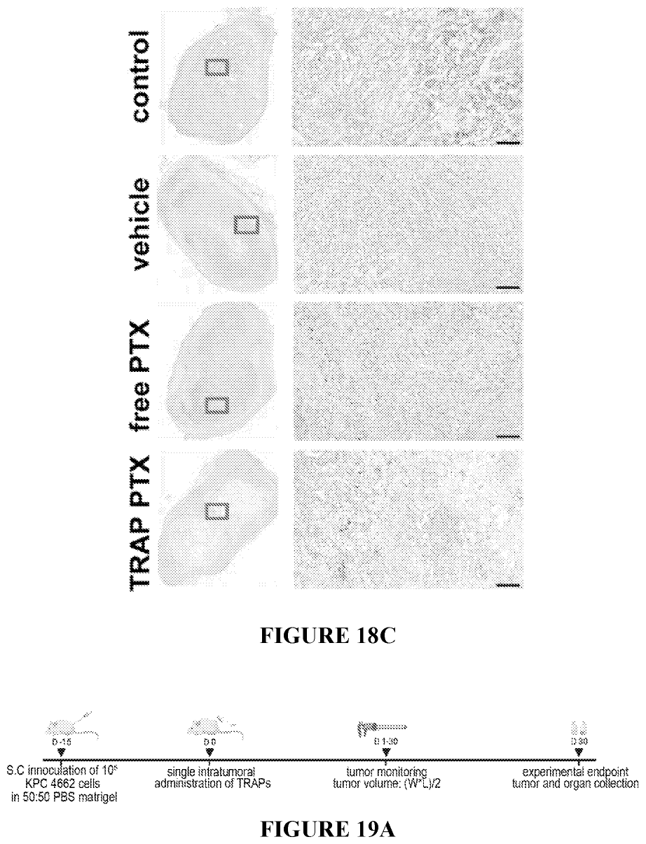

Paclitaxel TRAP induces strong apoptosis in vivo. To investigate whether intratumoral injection of TRAP paclitaxel has therapeutic efficacy in vivo, we studied paclitaxel-induced apoptosis in the fibrous, syngeneic KPC 4662 pancreatic adenocarcinoma mouse model. Once tumors reached approximately 100 mm³, animals were injected intratumorally with free paclitaxel, TRAP paclitaxel (PTX-NHS) or vehicle control. Although TRAP paclitaxel showed significantly improved aqueous solubility as compared to paclitaxel, a vehicle consisting of 50% NMP (42, 43) was used to allow direct comparison to the poorly soluble paclitaxel. Vehicle-treated and untreated tumors were used as negative controls. 72 hours after a single intratumoral injection, tumors were collected, fixed, sectioned, and stained for cleaved caspase 3 (CC-3), a marker for apoptosis that plays a central role in early events of cell death (44). For quantification of apoptosis, whole tissue sections were scanned to detect CC-3 positive areas and quantified to assess the percent of whole tumor area undergoing apoptosis. Tumor sections from treated groups showed large areas of diffuse CC-3 stain mixed with secondary necrosis. Sections from untreated controls showed central CC-3 positive region and minimal necrosis. Quantification of apoptosis was performed by computationally separating CC-3 stained from hematoxylin stained areas (45). Roughly 10% of total area from untreated tumors showed apoptotic signals. While both vehicle and free PTX treatment increased the apoptotic area (24% and 27%, respectively) neither reached statistical significance as compared to untreated group. In contrast, tumors treated with TRAP paclitaxel demonstrated significant increase in apoptotic staining (38% of total tumor) as compared to untreated control and vehicle control groups. To further assess the paclitaxel induced apoptosis and to separate apoptosis from vehicle-induced necrosis, all sections were quantified for viable areas showing uniform cell staining and architecture, apoptotic areas showing CC-3 positive staining, and necrotic areas showing presence of cellular debris and loss of tumor architecture (FIGS. 18A-18C) by measuring the areas based on color saturations (46). In this analysis, over 90% of total tumor area in the untreated controls was viable, with less than 5% of apoptotic and necrotic, each. Treatment with either vehicle, free paclitaxel, or TRAP paclitaxel, induced roughly similar levels of necrosis (˜30% of tumor area), demonstrating the contribution of vehicle-ablation to induce local necrotic cell death. However, intratumoral administration of TRAP paclitaxel increased the percent apoptotic area by 3-5 fold over administration of vehicle or free paclitaxel.

Taken together, significantly higher apoptosis and significantly lower viability within the tumors treated with TRAP paclitaxel demonstrate the contribution of TRAP-mediated controlled paclitaxel release to maintain higher paclitaxel concentrations in the tumor ECM for sustained therapeutic benefits.

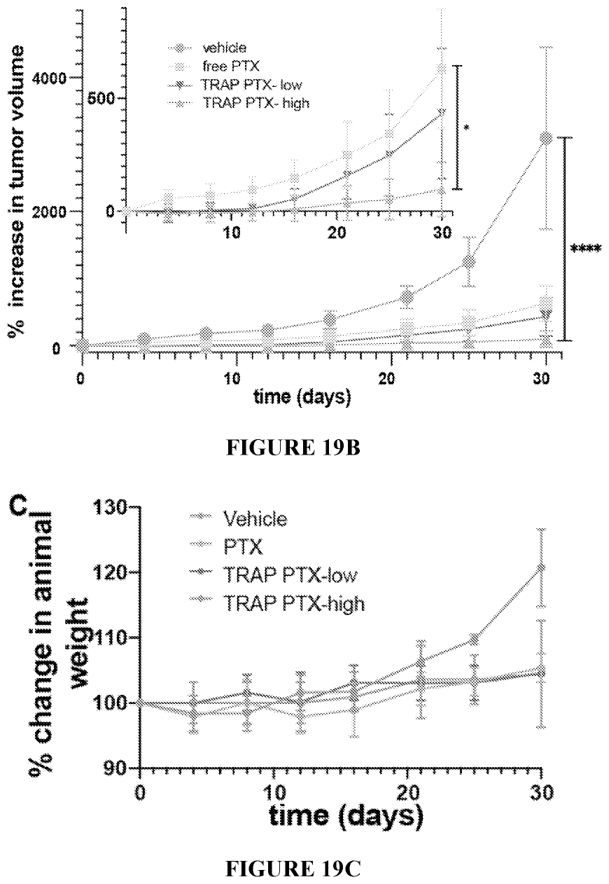

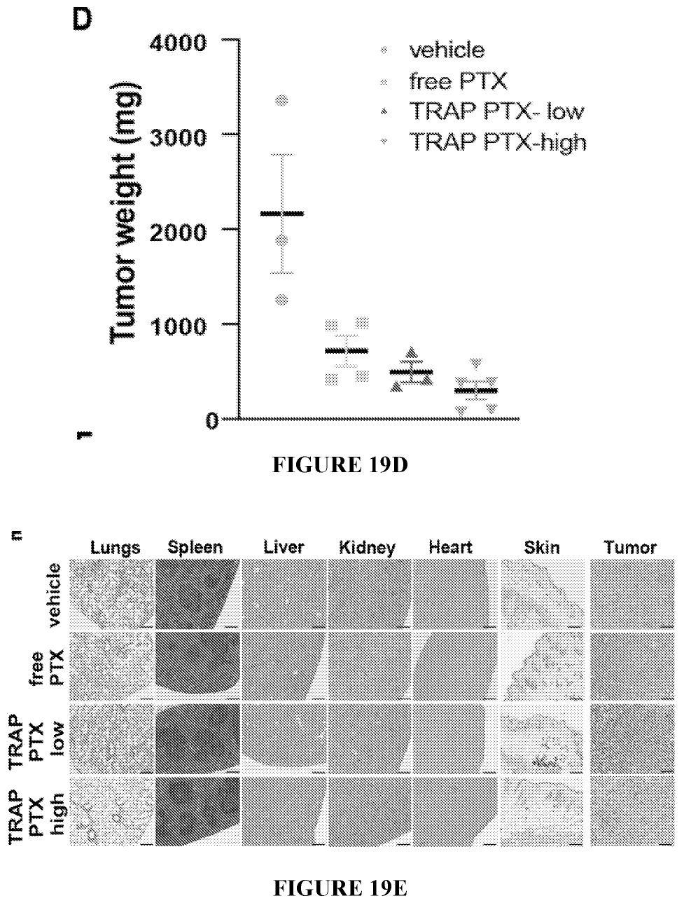

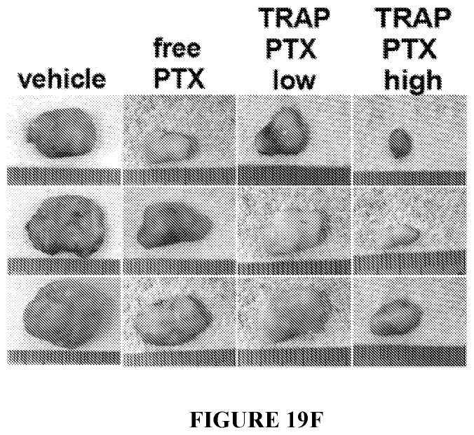

Paclitaxel TRAP promotes anti-cancer efficacy. Since TRAP paclitaxel induced significantly more apoptotic tumor cell death than free paclitaxel control, we tested its antitumor efficacy in comparison to injection of free paclitaxel and vehicle in a syngeneic pancreatic tumor model. Animals bearing KPC 4662 murine pancreatic tumors (˜100 mm³ in volume) were randomly divided between four groups to receive intratumoral treatment of vehicle, free paclitaxel, or TRAP paclitaxel. Since TRAP-paclitaxel is more soluble than free paclitaxel, it could be given at two doses, a “low” dose for direct comparison to free paclitaxel and a “high” dose, closer to TRAP paclitaxel's solubility limit. Tumor volume and animal weight were monitored over 30 days (FIG. 19A). Animals receiving either dose of PTX-sNHS showed significant tumor growth suppression as compared to vehicle and free paclitaxel control groups. In animals receiving the “low” TRAP paclitaxel dose significance lasted through day 15, whereas the higher dosage of TRAP paclitaxel showed continuous tumor inhibition compared to both negative control and free paclitaxel groups (FIG. 19B), highlighting the advantage of TRAP paclitaxel to provide sustained intratumoral paclitaxel at therapeutic concentrations to reduce tumor burden. Animal weight monitoring showed no apparent body weight reduction (FIG. 19C), suggesting all animals were able to tolerate intratumoral injections and treatments well. On day 30, all animals were euthanized to collect tumors and main organs. Mice treated with the “high” dose of TRAP paclitaxel carried lower tumor weight as compared to other groups (FIG. 19D). A blinded histological analysis by a veterinary histopathologist was performed to evaluate presence of systemic toxicity and suggested no apparent differences in the organs (lungs, hearts, spleens, livers, kidneys and skin) among different treated groups (FIG. 19E). Since vehicle was observed to induce local necrosis, the anti-tumor impact of vehicle was compared directly to saline controls. A small, but not statistically significant difference in tumor growth was observed in animals receiving intratumoral vehicle versus saline, suggesting little contribution of NMP in the vehicle to tumor growth inhibition. In addition, intratumoral NMP was tested for systemic toxicity by comparing mice receiving intratumoral vehicle injections to untreated control mice. Histological analysis by a blinded veterinary histopathologist found no differences between the vehicle-injected and untreated groups.

Taken together, continuous paclitaxel presentation at the tumor made possible by intratumorally anchored paclitaxel depots provided superior antitumor efficacy without off-target toxicity. Further improvements to the TRAP technology, including optimization of intratumoral infusion parameters, identification of alternative TRAP chemistries, and use of additional intratumoral infusion sites could provide even better anti-tumor efficacy and animal survival.

### REFERENCES

1. J. B. Wolinsky, Y. L. Colson, M. W. Grinstaff, Local drug delivery strategies for cancer treatment: gels, nanoparticles, polymeric films, rods, and wafers. J. Control. Release 159, 14-26 (2012).
2. A. Wadee, et al., Recent advances in the design of drug-loaded polymeric implants for the treatment of solid tumors. Expert Opin. Drug Deliv. 8, 1323-1340 (2011).
3. H.-S. Kim, J. Yang, K. Kim, U. S. Shin, Biodegradable and injectable hydrogels as an immunosuppressive drug delivery system. Mater. Sci. Eng. C Mater. Biol. Appl. 98, 472-481 (2019).
4. M. Szulc, A. Zakrzewska, J. Zborowski, Local drug delivery in periodontitis treatment: A review of contemporary literature. Dent Med Probl 55, 333-342 (2018).
5. J. M. Stewart, B. G. Keselowsky, Combinatorial drug delivery approaches for immunomodulation. Adv. Drug Deliv. Rev. 114, 161-174 (2017).
6. J. Azzi, et al., Targeted Delivery of Immunomodulators to Lymph Nodes. Cell Rep. 15, 1202-1213 (2016).
7. S. Pandey, N. Rai, P. Rawat, F. J. Ahmad, S. Talegaonkar, Nanofacilitated synergistic treatment for rheumatoid arthritis: A ‘three-pronged’ approach. Medical Hypotheses 92, 44-47 (2016).
8. R. De Souza, P. Zahedi, C. J. Allen, M. Piquette-Miller, Polymeric drug delivery systems for localized cancer chemotherapy. Drug Deliv. 17, 365-375 (2010).
9. Z. Sun, C. Song, C. Wang, Y. Hu, J. Wu, Hydrogel-Based Controlled Drug Delivery for Cancer Treatment: A Review. v Mol. Pharm. 17, 373-391 (2020).
10. Z. Mohtashami, Z. Esmaili, M. A. Vakilinezhad, E. Seyedjafari, H. Akbari Javar, Pharmaceutical implants: classification, limitations and therapeutic applications. Pharm. Dev. Technol. 25, 116-132 (2020).
11. C. Y. Lim, et al., Succinimidyl ester surface chemistry: implications of the competition between aminolysis and hydrolysis on covalent protein immobilization. Langmuir 30, 12868-12878 (2014).
12. S. Kalkhof, A. Sinz, Chances and pitfalls of chemical cross-linking with amine-reactive N-hydroxysuccinimide esters. Anal. Bioanal. Chem. 392, 305-312 (2008).
13. M. R. Adams, C. T. Moody, J. L. Sollinger, Y. Brudno, Extracellular-matrix-anchored click motifs for specific tissue targeting. Mol. Pharm. 17, 392-403 (2020).
14. M. O. Durymanov, A. A. Rosenkranz, A. S. Sobolev, Current Approaches for Improving Intratumoral Accumulation and Distribution of Nanomedicines. Theranostics 5, 1007-1020 (2015).
15. P. E. Oberstein, K. P. Olive, Pancreatic cancer: why is it so hard to treat? Therap. Adv. Gastroenterol. 6, 321-337 (2013).
16. E. van Veldhuisen, et al., Locally Advanced Pancreatic Cancer: Work-Up, Staging, and Local Intervention Strategies. Cancers 11 (2019).
17. P. Hammel, et al., Effect of chemoradiotherapy vs chemotherapy on survival in patients with locally advanced pancreatic cancer controlled after 4 months of gemcitabine with or without erlotinib. JAMA 315, 1844 (2016).
18. T. Conroy, et al., Current standards and new innovative approaches for treatment of pancreatic cancer. Eur. J. Cancer 57, 10-22 (2016).
19. L. Miao, C. M. Lin, L. Huang, Stromal barriers and strategies for the delivery of nanomedicine to desmoplastic tumors. J. Control. Release 219, 192-204 (2015).
20. C. R. Drifka, et al., Highly aligned stromal collagen is a negative prognostic factor following pancreatic ductal adenocarcinoma resection. Oncotarget 7, 76197-76213 (2016).
21. P. P. Adiseshaiah, R. M. Crist, S. S. Hook, S. E. McNeil, Nanomedicine strategies to overcome the pathophysiological barriers of pancreatic cancer. Nat. Rev. Clin. Oncol. 13, 750-765 (2016).
22. M. Brunner, et al., Current Clinical Strategies of Pancreatic Cancer Treatment and Open Molecular Questions. Int. J Mol. Sci. 20 (2019).
23. R. Shah, K. T. Ostapoff, B. Kuvshinoff, S. N. Hochwald, Ablative Therapies for Locally Advanced Pancreatic Cancer. Pancreas 47, 6-11 (2018).
24. K. Shi, et al., Sustained co-delivery of gemcitabine and cis-platinum via biodegradable thermo-sensitive hydrogel for synergistic combination therapy of pancreatic cancer. Nano Res. 12, 1389-1399 (2019).
25. H. Okino, R. Maeyama, T. Manabe, T. Matsuda, M. Tanaka, Trans-tissue, sustained release of gemcitabine from photocured gelatin gel inhibits the growth of heterotopic human pancreatic tumor in nude mice. Clin. Cancer Res. 9, 5786-5793 (2003).
26. J. P. Smith, et al., Intratumoral chemotherapy with a sustained-release drug delivery system inhibits growth of human pancreatic cancer xenografts. Anticancer Drugs 6, 717-726 (1995).
27. Y. Ramot, et al., Preclinical Safety Evaluation in Rats of a Polymeric Matrix Containing an siRNA Drug Used as a Local and Prolonged Delivery System for Pancreatic Cancer Therapy. Toxicol. Pathol. 44, 856-865 (2016).
28. L. Indolfi, et al., A tunable delivery platform to provide local chemotherapy for pancreatic ductal adenocarcinoma. Biomaterials 93, 71-82 (2016).
29. H.-G. Yi, et al., A 3D-printed local drug delivery patch for pancreatic cancer growth suppression. J. Control. Release 238, 231-241 (2016).
30. E. Jun, et al., Synergistic effect of a drug loaded electrospun patch and systemic chemotherapy in pancreatic cancer xenograft. Sci. Rep. 7, 12381 (2017).
31. S. Bang, et al., Molecular mechanism of local drug delivery with Paclitaxel-eluting membranes in biliary and pancreatic cancer: new application for an old drug. Gastroenterol. Res. Pract. 2015, 568981 (2015).
32. J. P. Smith, et al., Drug retention and distribution after intratumoral chemotherapy with fluorouracil/epinephrine injectable gel in human pancreatic cancer xenografts. Cancer Chemother. Pharmacol. 44, 267-274 (1999).
33. A. A. Exner, G. M. Saidel, Drug-eluting polymer implants in cancer therapy. Expert Opin. Drug Deliv. 5, 775-788 (2008).
34. Y. Miyasaka, et al., Neoadjuvant chemotherapy with gemcitabine plus nab-paclitaxel for borderline resectable pancreatic cancer potentially improves survival and facilitates surgery. Ann. Surg. Oncol. 26, 1528-1534 (2019).
35. R. Panchagnula, Pharmaceutical aspects of paclitaxel. Int. J. Pharm. 172, 1-15 (1998).
36. A. H. Kyle, L. A. Huxham, D. M. Yeoman, A. I. Minchinton, Limited tissue penetration of taxanes: a mechanism for resistance in solid tumors. Clin. Cancer Res. 13, 2804-2810 (2007).
37. A. E. Massey, et al., Next-generation paclitaxel-nanoparticle formulation for pancreatic cancer treatment. Nanomedicine 20, 102027 (2019).
38. S. Y. Min, et al., Facile one-pot formulation of TRAIL-embedded paclitaxel-bound albumin nanoparticles for the treatment of pancreatic cancer. Int. J. Pharm. 494, 506-515 (2015).
39. Y. Mao, X. Li, G. Chen, S. Wang, Thermosensitive hydrogel system with paclitaxel liposomes used in localized drug delivery system for in situ treatment of tumor: Better antitumor efficacy and lower toxicity. v J. Pharm. Sci. 105, 194-204 (2016).
40. K. Matthes, et al., EUS-guided injection of paclitaxel (OncoGel) provides therapeutic drug concentrations in the porcine pancreas (with video). Gastrointest. Endosc. 65, 448-453 (2007).
41. Q. Fu, et al., Programmed Hydrolysis in Designing Paclitaxel Prodrug for Nanocarrier Assembly. Sci. Rep. 5, 12023 (2015).
42. R. Sanghvi, R. Narazaki, S. G. Machatha, S. H. Yalkowsky, Solubility improvement of drugs using N-methyl pyrrolidone. AAPS PharmSciTech 9, 366-376 (2008).
43. A. Jouyban, M. A. A. Fakhree, A. Shayanfar, Review of pharmaceutical applications of N-methyl-2-pyrrolidone. J. Pharm. Pharm. Sci. 13, 524-535 (2010).
44. P.-F. Liu, et al., Expression levels of cleaved caspase-3 and caspase-3 in tumorigenesis and prognosis of oral tongue squamous cell carcinoma. PLoS One 12, e0180620 (2017).
45. L. J. Janke, J. M. Ward, P. Vogel, Classification, Scoring, and Quantification of Cell Death in Tissue Sections. Vet. Pathol. 56, 33-38 (2019).
46. M. Krajewska, et al., Image Analysis Algorithms for Immunohistochemical Assessment of Cell Death Events and Fibrosis in Tissue Sections. Journal of Histochemistry & Cytochemistry 57, 649-663 (2009).
47. N. Renier, et al., iDISCO: a simple, rapid method to immunolabel large tissue samples for volume imaging. Cell 159, 896-910 (2014).
48. L. Shan, et al., A paclitaxel-conjugated adenovirus vector for targeted drug delivery for tumor therapy. Biomaterials 33, 146-162 (2012).

The compositions, systems, and methods of the appended claims are not limited in scope by the specific devices, systems, and methods described herein, which are intended as illustrations of a few aspects of the claims. Any devices, systems, and methods that are functionally equivalent are intended to fall within the scope of the claims. Various modifications of the devices, systems, and methods in addition to those shown and described herein are intended to fall within the scope of the appended claims. Further, while only certain representative devices, systems, and method steps disclosed herein are specifically described, other combinations of the devices, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A method for delivering an active agent to a target tissue in a subject, the method comprising:
contacting the target tissue with a therapeutically effective amount of a compound defined by Formula I
X-L¹-CM¹ Formula I
wherein
X represents a tissue binding moiety;
L¹ is absent, or represents a linking group; and
A represents an active agent
wherein the tissue binding moiety comprises a functional group capable of chemically reacting with the target tissue to form a covalent bond.

2. The method of claim 1, wherein the tissue binding moiety comprises a functional group capable of chemically reacting with a functional group in a peptide to form a covalent bond.

3. The method of claim 2, wherein the tissue binding moiety comprises a functional group capable of chemically reacting with an amine group in a peptide to form a covalent bond.

4. The method of claim 3, wherein the tissue binding moiety comprises a sulfo-hydroxysuccinimidyl (sNHS) group.

5. The method of claim 2, wherein the tissue binding moiety comprises a functional group capable of chemically reacting with a thiol group in a peptide to form a covalent bond.

6. The method of claim 5, wherein the tissue binding moiety comprises a maleimide group.

7. The method of claim 1, wherein the tissue binding moiety binds with the extracellular matrix in the target tissue.

8. The method of claim 7, wherein the tissue binding moiety comprises a functional group capable of chemically reacting with the extracellular matrix in the target tissue to form a covalent bond.

9. The method of claim 8, wherein the tissue binding moiety comprises a functional group capable of chemically reacting with a protein present in the extracellular matrix in the target tissue to form a covalent bond.

10. The method of claim 9, wherein the protein comprises collagen.

11. The method of claim 1, wherein L¹ is absent.

12. The method of claim 1, wherein L¹ represents a cleavable linker, such as a hydrolytically cleavable linker, a photocleavable linker, or an enzymatically cleavable linker.

13. The method of claim 1, wherein the active agent comprises a diagnostic agent.

14. The method of claim 1, wherein the active agent comprises a therapeutic agent.

15. The method of claim 14, wherein the therapeutic agent comprises an anti-cancer drug, a drug that promotes wound healing, a drug that promotes vascularization, a drug that treats or prevents infection, a drug that prevent restenosis, a drug that reduces macular degeneration, a drug that prevents immunological rejection, a drug that prevents thrombosis, or a drug that treats inflammation.

16. The method of claim 15, wherein the therapeutic agent comprises an anti-cancer drug.

17. The method of claim 15, wherein the therapeutic agent comprises a drug that promotes wound healing.

18. The method of claim 15, wherein the therapeutic agent comprises a drug that promotes vascularization or a drug that prevents restenosis.

19. The method of claim 15, wherein the therapeutic agent comprises a drug that treats or prevents infection.

20. The method of claim 1, wherein the target tissue comprises a solid tumor.

21. The method of claim 1, wherein contacting the target tissue the therapeutically effective amount of the compound defined by Formula I comprises injecting the therapeutically effective amount of the compound defined by Formula I into the target tissue.

22. A method of maintaining or reducing the size of a tumor in a subject in need thereof, comprising the steps of:
(i) injecting into the tumor a therapeutically effective amount of a compound defined by Formula I
X-L¹-CM¹ Formula I
wherein
X represents a tissue binding moiety;
L¹ is absent, or represents a linking group; and
A represents an active agent
thereby maintaining or reducing the size of the tumor in the subject
wherein the tissue binding moiety comprises a functional group capable of chemically reacting with tissue to form a covalent bond.
